(19) United States
(12) Patent Application Publication
(10) Pub. No.: US 2024/0207213 A1
(43) Pub. Date: Jun. 27, 2024

(54) METHODS AND COMPOSITIONS FOR TREATING DISORDERS WITH ORAL SYSTEMICALLY BIOAVAILABLE BUTYRATE CONJUGATES

(71) Applicant: THE UNIVERSITY OF CHICAGO, Chicago, IL (US)

(72) Inventors: Jeffrey HUBBELL, Chicago, IL (US); Shijie CAO, Chicago, IL (US); Michal RACZY, Chicago, IL (US); Elyse WATKINS, Chicago, IL (US); Erica BUDINA, Chicago, IL (US)

(21) Appl. No.: 18/556,006

(22) PCT Filed: Apr. 18, 2022
(86) PCT No.: PCT/US22/71775

§ 371 Date: Oct. 18, 2023

Related U.S. Application Data
(60) Provisional application No. 63/176,749, filed on Apr. 19, 2021.

(51) Int. Cl. A61K 31/221; A61K 9/00; A61P 37/00

(52) U.S. Cl. CPC A61K 31/221; A61K 9/0053; A61P 37/00

(57) ABSTRACT

Described herein are novel methods of treating disorders using oral, systemically bioavailable forms of butyrate. Aspects of the disclosure relate to a method for treating a disorder in a subject comprising administering the oral, systemically bioavailable composition to the subject, wherein the disorder is selected from inflammatory bowel disease (IBD), multiple sclerosis (MS), a neuroinflammatory condition, food allergies, rheumatoid arthritis (RA), asthma, celiac disease, non-alcoholic steatohepatitis (NASH), diabetes, Alzheimer's disease (AD), inflammaging, beta-hemoglobinopathies, vascular inflammatory conditions, atopic dermatitis and psoriasis, pulmonary fibrosis, and systemic sclerosis.

### CROSS-REFERENCE TO RELATED APPLICATIONS

This application claims the benefit of priority of U.S. Provisional Patent Application No. 63/176,749 filed Apr. 19, 2021, which is hereby incorporated by reference in its entirety.

### BACKGROUND

### I. Field of the Invention

This invention relates to the field of molecular biology and medicine.

### II. Background

Butyrate is a short chain fatty acid derived from the microbial fermentation of dietary fibers in the colon. In the intestine, butyrate is a primary energy source for colonocytes and maintains intestinal homeostasis (3). It has been shown to be necessary for protection of intestinal barrier function through facilitating tight junction assembly (10, 11). Butyrate is an epigenetic substance classified as a histone deacetylate (HDAC) inhibitor, that can mold chromatic structures and regulate gene expression with stability and define cell's identity, function, and adaptation without changing the DNA sequence (12, 13). Through HDAC inhibition, butyrate has been shown to suppress nuclear factor κB (NFκB) activation, inhibit production of interferon γ (IFNγ) and upregulate peroxisome proliferator-activated receptor γ (PPARγ) (1). Butyrate exerts broad anti-inflammatory activity by affecting immune cell migration, adhesion, cytokine expression, proliferation, activation and apoptosis (14). Aside from HDAC inhibition, butyrate can exert anti-inflammatory effects on immune cells, such us dendritic cells and Tregs, via signaling through specific G-protein coupled receptors (GPRs): GPR41, GPR43 and GPR109A (15). Taken together, these properties of butyrate have important implications in the development of therapeutic strategies, especially for the treatment of inflammatory diseases, allergies, and auto-immune diseases. However, oral supplementation of sodium butyrate has been a challenge because butyrate, even with enteric coating or encapsulation, possesses a foul and lasting odor and taste. In addition, butyrate is not absorbed in the part of the gut where it can have a therapeutic effect and is metabolized too rapidly to maintain a pharmacologic effect. Other possible routes of butyrate administration, e.g. intrarectal delivery and continuous intravenous infusion, are often unacceptable to patients with chronic disorders (16-19). New delivery methods of butyrate, including prodrugs that can enhance its systemic bioavailability and pharmacokinetics, are needed for butyrate to make an impact as anti-inflammatory treatment.

### SUMMARY

The current disclosure provides for novel methods of treating disorders using oral, systemically bioavailable forms of butyrate. Aspects of the disclosure relate to a method for treating a disorder in a subject comprising administering an oral composition comprising a compound selected from

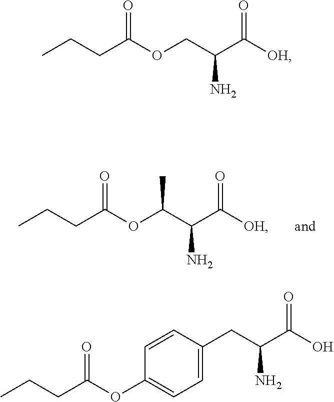 [SMILES: CCCC(=O)OC[C@H](N)C(=O)O ; CCCC(=O)O[C@@H](C)[C@H](N)C(=O)O ; "and" ; CCCC(=O)Oc1ccc(C[C@H](N)C(=O)O)cc1]

to the subject, wherein the disorder is inflammatory bowel disease (IBD), multiple sclerosis (MS), a neuroinflammatory condition, food allergies, rheumatoid arthritis (RA), asthma, celiac disease, non-alcoholic steatohepatitis (NASH), diabetes, Alzheimer's disease (AD), inflammaging, beta-hemoglobinopathies, vascular inflammatory conditions, atopic dermatitis and psoriasis, pulmonary fibrosis, or systemic sclerosis.

Aspects of the disclosure also relate to the use of compositions and compounds of the disclosure for use in a method for treating inflammatory bowel disease (IBD), multiple sclerosis (MS), a neuroinflammatory condition, food allergies, rheumatoid arthritis (RA), asthma, celiac disease, non-alcoholic steatohepatitis (NASH), diabetes, Alzheimer's disease (AD), inflammaging, beta-hemoglobinopathies, vascular inflammatory conditions, atopic dermatitis and psoriasis, pulmonary fibrosis, or systemic sclerosis in a subject. Further aspects relate to the use of the compositions and compounds of the disclosure for use in the preparation for a medicament for treating a disorder in a subject, wherein the disorder is selected from inflammatory bowel disease (IBD), multiple sclerosis (MS), a neuroinflammatory condition, food allergies, rheumatoid arthritis (RA), asthma, celiac disease, non-alcoholic steatohepatitis (NASH), diabetes, Alzheimer's disease (AD), inflammaging, beta-hemoglobinopathies, vascular inflammatory conditions, atopic dermatitis and psoriasis, pulmonary fibrosis, and or systemic sclerosis.

In some aspects, the compound comprises

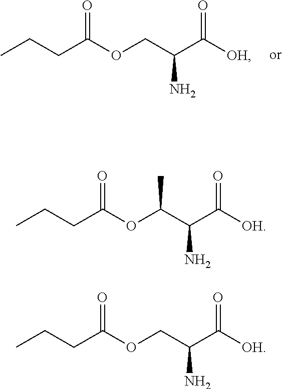

In some aspects, the compound comprises In some aspects, the methods exclude treatment of inflammatory bowel disease (IBD), multiple sclerosis (MS), a neuroinflammatory condition, food allergies, rheumatoid arthritis (RA), asthma, celiac disease, non-alcoholic steatohepatitis (NASH), diabetes, Alzheimer's disease (AD), inflammaging, beta-hemoglobinopathies, vascular inflammatory conditions, atopic dermatitis and psoriasis, pulmonary fibrosis, or systemic sclerosis.

In some aspects, the methods relate to a method for treating multiple sclerosis in a subject comprising administering an oral composition comprising

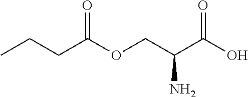

to the subject. The subject may be a human subject. The total daily dose administered to the subject may be 25 or 50 mg/kg. In some aspects, the subject is administered two doses per day, wherein the total daily dose is 25 or 50 mg/kg. In some aspects, the subject is administered two doses per day, wherein the total daily dose is 50 mg/kg.

In some aspects, the administered compound excludes

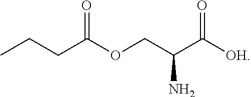

In some aspects, the administered compound excludes

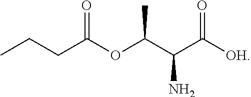

In some aspects, the administered compound excludes

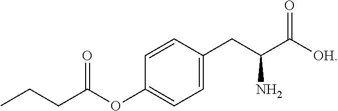 [SMILES: CCCC(=O)Oc1ccc(C[C@H](N)C(=O)O)cc1]

The diabetes may be further defined as Type 1 diabetes. In some aspects, the diabetes is further defined as Type 2 diabetes. The beta-hemoglobinopathies may be thalassemia or sickle cell disease. The vascular inflammatory condition may be atherosclerosis. In some aspects the vascular inflammatory condition is restenosis. The neuroinflammatory condition may comprise diabetic peripheral neuropathy, neuroinflammation, neuroinflammation associated with traumatic brain injury, neuroinflammation associated with aging, neuroinflammation associated with spinal cord injury, neuroinflammation associated with infection, neuromyelitis optica, MS, Alzheimer's disease, or Parkinson's disease.

The compound in the methods of the disclosure may be further defined as being labile in vivo. The methods of the disclosure may comprise administration of a systemically bioavailable composition. The compound may be one that demonstrates significant biodistribution to the plasma, liver, mesenteric lymph nodes, brain, spinal cord, lung, and spleen over a control, wherein the control comprises sodium butyrate. For example, the measured or experimentally expected concentration in the plasma, liver, mesenteric lymph nodes, brain, spinal cord, lung, and spleen may be at least or may be at most about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, or 550 (or any derivable range therein) nM or μg/g.

The compound of the disclosure may be administered as a daily dose to the subject. The total amount of compound, over a 24 hour period (or on average of over a 24-hour period) may be, be at least, or be at most about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) mg or grams. In some aspects, the composition comprises less than 4 grams of the compound in the daily dose. The term “daily dose” refers to the total amount of compound administered or intended to be administered in a 24-hour period. Therefore, the subject may be administered multiple doses in a day, but the total daily dose is the total amount of compound administered in a 24-hour period.

In some aspects, the composition comprises 2-8 grams of the compound in the daily dose. In some aspects, the composition comprises 3-6 grams of the compound in the daily dose. In some aspects, the composition comprises 6 grams of the compound in the daily dose. In some aspects, the composition comprises 20-150 mg/kg of the compound in the daily dose. In some aspects, the composition comprises 50-100 mg/kg of the compound in the daily dose. In some aspects, the composition comprises 100 mg/kg of the compound in the daily dose.

The daily dose need not be administered in one composition at one time during the day, but may be in multiple, such as 2, 3, 4, 5, or 6 different compositions administered at different or at the same time of the day. The compositions may be administered at least 10 hours apart, at least 12 hours apart, or at time period of, a time period of at least, or a time period of at most 1, 2, 3, 4, 5, 6, 7, 8, 9, 10, 11, 12, 13, 14, 15, 16, 17, 18, 19 or 20 hours apart. In some aspects, the amount of compound administered is at least or is at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) mg or grams and is administered every day, twice a day, three times a day, four times a day, five times a day, six times a day, every other day, once a week, twice a week, three times a week, four times a week, five times a week, or six times a week (or 5 any derivable range therein) for at least or for at most 1 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or 1, 2, 3, 4, 5, 6, 7, 8, 9, or 10 years (or any derivable range therein). In some aspects, the daily dose is administered for at least 30 days.

In some aspects, the amount of compound administered is at least or is at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 (or any derivable range therein) mg/kg or grams/kg and is administered every day, every other day, once a week, twice a week, three times a week, four times a week, five times a week, or six times a week (or any derivable range therein) for at least or for at most 1 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or 1, 2, 3, 4, 5, 6, 7, 8, 9, or 10 years (or any derivable range therein). In some aspects, the daily dose is administered for at least 30 days. In some aspect, the compound is administered daily for an indefinite amount of time.

The subject may be a mammal, human, dog, cat, mouse, pig, horse, rat, or rabbit. The subject in the methods of the disclosure may be a human subject. The subject may be at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 years old (or any derivable range therein). The subject may be 18 years old or more. In some aspects, the subject is 50 years old or more.

The administration of the composition may alleviate one or more symptoms of the disorder. In some aspects, administration of the composition reduces inflammation in the subject.

The composition may comprise a solution. The composition may also be comprised of a tablet. The tablet may comprise enteric coating and/or encapsulation of the compound. In some aspects, enteric coating and/or encapsulation of the compound is excluded. Additional formulations may be suitable for oral administration. “Oral administration” as used herein refers to any form of delivery of a therapeutic agent or composition thereof to a subject wherein the agent or composition is placed in the mouth of the subject, whether or not the agent or composition is swallowed. Thus, “Oral administration” includes buccal and sublingual as well as esophageal administration. Absorption of the agent can occur in any part or parts of the gastrointestinal tract including the mouth, esophagus, stomach, duodenum, ileum and colon. Oral formulations include such typical excipients as, for example, pharmaceutical grades of mannitol, lactose, starch, magnesium stearate, sodium saccharine, cellulose, magnesium carbonate and the like. The compositions may take the form of solutions, suspensions, tablets, pills, capsules, sustained release formulations or powders. In some instances, the methods of the disclosure may exclude administration of the compounds of the disclosure in sustained release formulations or powders. In some aspects, oral administration may exclude buccal, sublingual, or esophageal administration. Oral formulations may exclude such typical excipients as, for example, pharmaceutical grades of mannitol, lactose, starch, magnesium stearate, sodium saccharine, cellulose, magnesium carbonate and the like. In some aspects, the compound is comprised in a sachet as a powder and is mixed with a liquid just prior to administration. The liquid may comprise water.

In some aspects, the serum concentration of the compound 0-5 hours after administration is at least 50 nM. In some aspects, the serum concentration of the compound after 5, 10, 15, 20, 25, 30, 35, 40, 45, 50, or 55 minutes or 1, 2, 3, 4, 5, 6, 7, 8, 9, 10, 11, 12, 18, or 24 hours of administration is at least or is at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, or 550 nM (or any derivable range therein). The level of the compound in the liver, mesenteric lymph nodes, brain, spinal cord, and/or spleen is estimated to be at least 2 μg/g tissue. In some aspects, the level of the compound in the liver, mesenteric lymph nodes, brain, spinal cord, and/or spleen is estimated to be at least or to be at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/g tissue (or any derivable range therein). In some aspects, the level of the compound in the liver, mesenteric lymph nodes, brain, spinal cord, and/or spleen is estimated to be at least 10 μg/g tissue.

Throughout this application, the term “about” is used to indicate that a value includes the inherent variation of error for the measurement or quantitation method.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments and aspects discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments and aspects are discussed throughout this application. Any embodiment discussed with respect to one aspect of the disclosure applies to other aspects of the disclosure as well and vice versa.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the ¹H NMR spectrum of Serine Butyrate (500 MHz, DMSO-d₆).

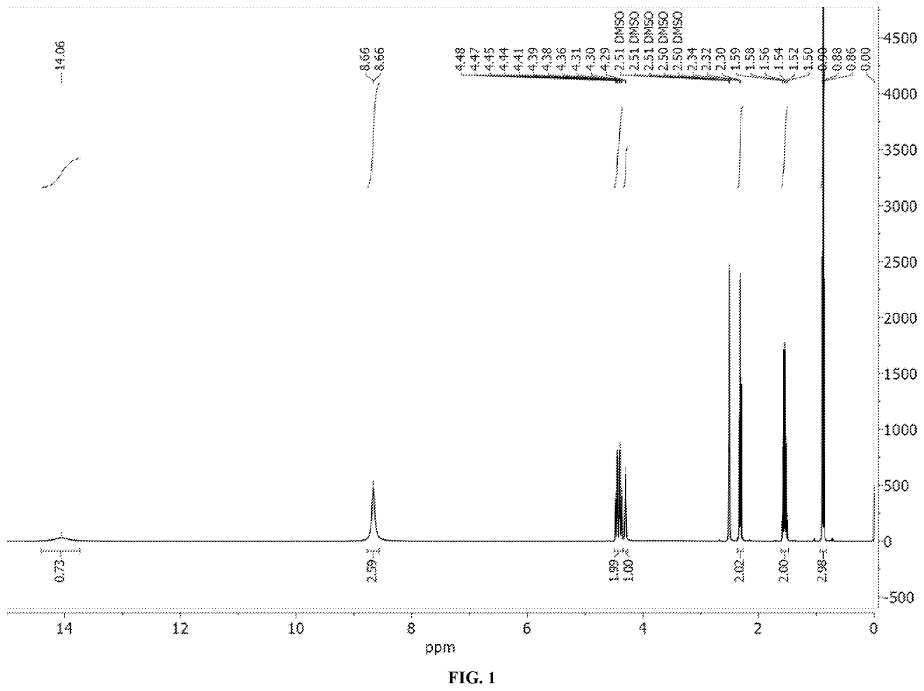

FIG. 2 shows the ¹H NMR spectrum of Threonine Butyrate (500 MHz, DMSO-d₆).

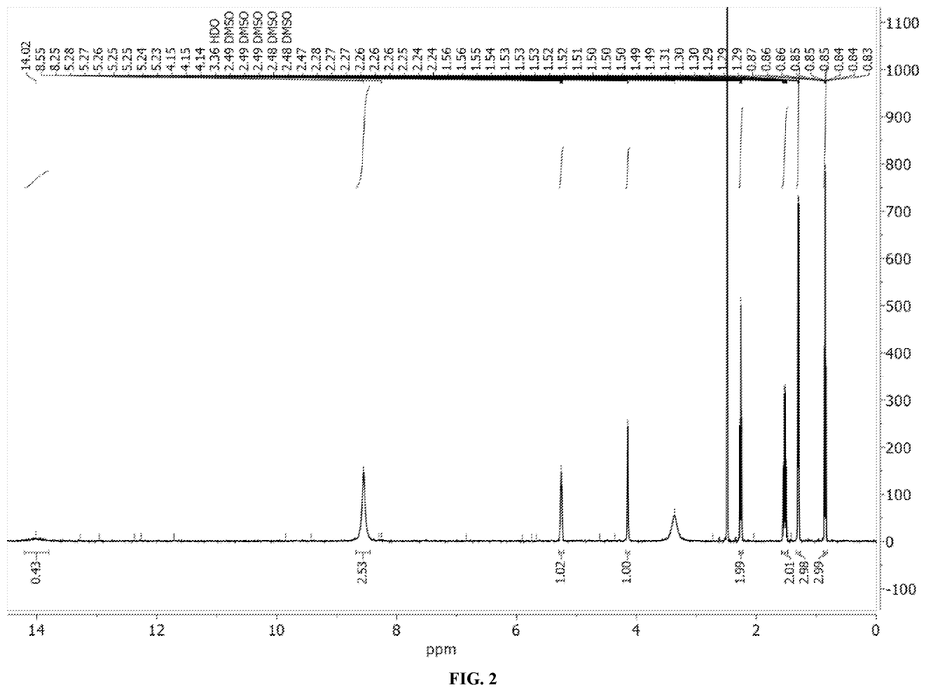

FIG. 3 shows the ¹H NMR spectrum of Tyrosine Butyrate (500 MHz, DMSO-d₆).

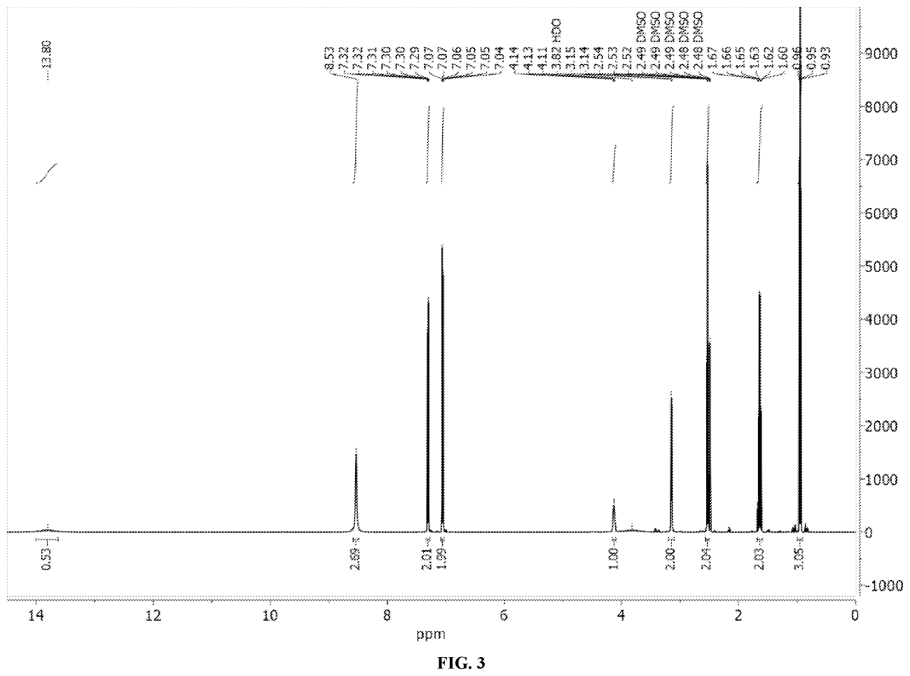

FIG. 4A-G shows the Biodistribution of sodium butyrate, serine butyrate and threonine butyrate after oral administration. The amount of butyrate quantified in the plasma (A), liver (B), mesenteric lymph nodes (mLNs) (C), brain (D), spinal cord (E), spleen (F), and lung (G). Blood was collected by cheek bleeding at 0.5 hr and 3 hr post oral gavage. Mice were sacrificed at 3 hr and 6 hr post oral gavage, and organs were collected for butyrate quantification. Spleens were collected only at 3 hr post-administration. Butyrate was derivatized with 3-nitrophenylhydrazine and quantified by LC-MS/MS. n=4-5.

FIG. 5A-D. Serine butyrate treatment attenuates the severity of experimental autoimmune encephalomyelitis (EAE). EAE was induced in C57BL/6 mice by injecting a MOG₃₅₋₅₅/CFA emulsion followed by pertussis toxin. Mice were twice daily orally administered with PBS (PBS+PBS, n=9) or serine butyrate (SerBut+SerBut, n=8), or with one dose of PBS and one dose of serine butyrate daily (PBS+SerBut, n=8). A. Disease progression is indicated by the clinical score. The areas under the curve were compared, and statistical analyses were performed using a one-way ANOVA with Dunnett's test. B. Clinical scores of three groups of mice at the day 20 post disease induction. Statistical analyses were performed using a one-way ANOVA with Dunnett's test. C. The probability that EAE clinical scores remained below 1.0 from three groups. Statistical analysis is performed using the Log-rank (Mantel-Cox) test comparing every two groups. D. Body weight changes of the mice. *p<0.05, **p<0.01.

FIG. 6A-C. Serine butyrate treatment inhibits leukocyte infiltration to the spinal cord and suppresses myeloid cells in the spinal cord. EAE was induced in C57BL/6 mice using MOG₃₅₋₅₅/CFA with pertussis toxin. Mice that were orally administered twice daily with PBS (PBS+PBS, n=9) or serine butyrate (SerBut+SerBut, n=8) were sacrificed at day 20, and the spinal cord were isolated and processed for immunostaining and analyzed by flow cytometry. A. The percentages of CD45+%, CD45+CD3+%, B220+%, CD11b+%, CD11b+CD11c−%, and F4/80+CD1 b+% of live cells in the spinal cord. B. The co-stimulatory molecule (CD80 and CD86) and MHCII expression on CD11c highly expressed cells in the spinal cord. C. The co-stimulatory molecule (CD80 and CD86) and MHCII expression on CD11b+CD11c− cells in the spinal cord. gMFI. geometrical mean fluorescent intensity. Statistical analyses were performed using either unpaired Student's t-test or Mann-Whitney test (based on the normality test). *p<0.05.

FIG. 7. Serine butyrate treatment suppresses CD11c^{hi} dendritic cells and F4/80+CD11b+ macrophages in the mesenteric LNs. The percentage of MHCII+% and geometric mean fluorescent intensity (gMFI) of CD11c^{hi} dendritic cells or F4/80+CD11b+ macrophages isolated from mesenteric LNs of EAE mice that treated with twice daily PBS or serine butyrate. Statistical analyses were performed using either unpaired Student's t-test or Mann-Whitney test (based on the normality test). *p<0.05, **p<0.01.

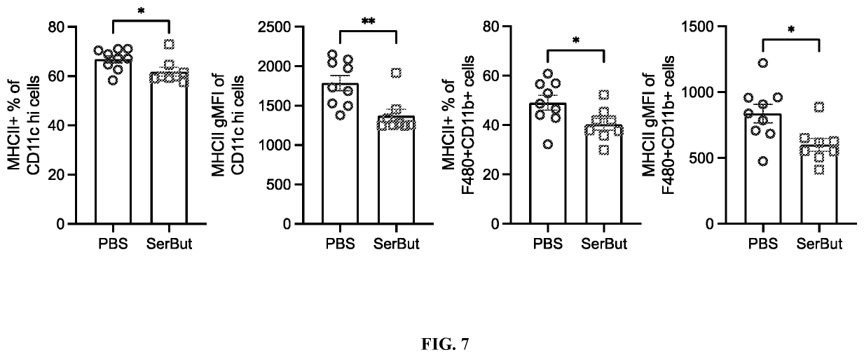

FIG. 8A-L. Serine butyrate treatment reduced Th17 cells in the spinal cord, and induced regulatory T cells in the spleen, CNS-draining LNs and mesenteric LNs of EAE mice. EAE was induced in C57BL/6 mice using MOG₃₅₋₅₅/CFA with pertussis toxin. Mice that were orally administered twice daily with PBS (PBS+PBS, n=9) or serine butyrate (SerBut+SerBut, n=8) were sacrificed at day 20, and the spinal cord, spleen, small intestine-draining mesenteric LNs, and CNS-draining iliac and cervical LNs were isolated and processed for immunostaining and analyzed by flow cytometry. The spinal cords were also homogenized and the supernatant were analyzed of IFNγ normalized by the total protein concentrations. A-D. The percentages of Foxp3+%, Foxp3+CD25+%, RORγt+% of CD4+ T cells in the spinal cord, and the concentration of IFNγ normalized by the total protein in the spinal cord homogenized supernatant. E-F. The percentages of Foxp3+%, Foxp3+CD25+% of CD4+ T cells in the spleen. G-I. The percentages of Foxp3+%, Foxp3+CD25+% of CD4+ T cells, and Foxp3+% of CD8+ T cells in the CNS-draining LNs. J-L. The percentages of Foxp3+%, Foxp3+CD25+% of CD4+ T cells, and Foxp3+% of CD8+ T cells in the mesenteric LNs. Statistical analyses were performed using either unpaired Student's t-test or Mann-Whitney test (based on the normality test). *p<0.05, **P<0.01.

### DETAILED DESCRIPTION OF THE INVENTION

Butyrate is a natural metabolite that is produced by microbes in the distal gut. Its concentration in the systemic circulation is low, because it is a major energy source for colonocytes and it thus substantially consumed there. Here, the inventors describe an approach by which to enable butyrate, or a precursor of butyrate, to escape metabolism in the gut, to be transported from the gut to the blood, and to distribute throughout the system, to thus be systemically bioactive. Our invention is to harness gut transport of amino acids into the blood by conjugating butyrate to amino acids via a link that can be labile in vivo, such as an ester link to amino acids that bear a hydroxyl group, including serine, threonine, and tyrosine. Serine butyrate and threonine butyrate are water soluble, and thus convenient, whereas tyrosine butyrate is not highly soluble and thus is less convenient. Many drugs further enter the brain and spinal cord poorly, due to the blood-brain barrier. The inventors further sought an approach whereby butyrate could cross the blood-brain barrier. The same approach of amino acid conjugation provided this, since amino acid transporters exist in the brain that provide amino acid transport from the blood into the cerebrospinal fluids that bathe the brain and spinal cord. Once in the system, both extracellularly and intracellularly the amino acid butyryl esters can by hydrolyzed nonenzymatically and enzymatically to produce butyrate to be biologically active. Here, the inventors teach treatment with amino acid derivatives of butyrate to treat inflammatory, immune and other conditions throughout the body, not limited to the gut, enabled by its systemic bioavailability, including brain and spinal cord. It is noted that the same molecular approach can be employed with other microbial metabolites, including acetate, propionate, valerate, and indole acetate.

### I. Definitions

“Individual, “subject,” and “patient” are used interchangeably and can refer to a human or non-human. The subject may be a mammal, a non-human primate, or a laboratory animal. Examples include human, mouse, rat, pig, horse, rabbit, and dog.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments and aspects described herein in the context of the term “comprising” may also be implemented in the context of the term “consisting of” or “consisting essentially of.”

### II. Pharmaceutical Compositions

The therapy provided herein may comprise administration of a combination of therapeutic agents, such as a first therapy and a second therapy. The therapies may be administered in any suitable manner known in the art. For example, the first and second treatment may be administered sequentially (at different times) or concurrently (at the same time). In some aspects, the first and second treatments are administered in a separate composition. In some aspects, the first and second treatments are in the same composition.

The methods of the disclosure relate to administration of compositions comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. The doses can be administered at multiple times during a day, and/or on multiple days, weeks, or months.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### III. Methods of Treatment

Compositions of the present disclosure may be administered to any individual who is suffering from a disorder described herein. The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance.

It is contemplated that methods may be applied to the disorders described herein and in sections A-O below.

### A. Food Allergies

The prevalence of food allergies has increased by as much as 50% over the past 20 years, particularly in developed countries. Genetics alone cannot explain such a dramatic rise in incidence over a single generation. Improvement in sanitation, dietary changes, and antibiotic use have been linked to increasing susceptibility to both allergic and autoimmune diseases. These changes may have altered the populations of commensal microbes that lead to a number of negative health effects. Short-chain fatty acids (SCFAs) derived from commensal bacteria, such as butyrate, may provide a promising therapeutic candidate for treating food allergies; however, oral delivery of butyrate to the small intestine (where food antigens are absorbed) has been a challenge because butyrate, even with enteric coating or encapsulation, possesses a foul and lasting odor and taste, and is metabolized too rapidly to achieve and maintain a pharmacologic effect. The provision of systemic butyrate through administration of compounds of the disclosure through oral administration dramatically increased the bioavailability of butyrate while it covered the unpleasant odor and smell of butyrate, thus could be good candidate for preventing and treating food allergies. The ability of the compounds of the disclosure to exit the gut and epithelium and enter the systemic circulation is key in providing butyrate to the depths of the gut wall and the gut-draining lymph nodes to prevent and treat food allergy.

### B. Inflammatory Bowel Diseases (IBD)

The IBD, mainly comprising ulcerative colitis (UC) and Crohn's disease (CD), affects approximately 3 million people in the USA and Europe, and the global number of prevalent cases of IBD has increased from 3.7 million in 1990 to more than 6.8 million in 2017. There is currently no therapeutic treatment that leads to a cure for IBD. The IBD can be caused by a complex interplay among genetic and environmental factors. Recently, dysbiosis of the gut microbiome has been recognized as an important causing factor to IBD, and is affected by the Western lifestyle. Some treatment strategies aim to correct gut microbial dysbiosis which promotes anti-inflammatory effects, but it is still challenging due to the metabolically complex cohort of bacterial interactions in the gut. Butyrate, which showed anti-inflammatory effects, maybe a practical approach. Oral administration of compounds of the disclosure might enhance the bioavailability of butyrate on the gut epithelial cells, as well as immune cells in the mesenteric lymph nodes, where the inventors have demonstrated high level of butyrate there through oral gavage of SerBut or ThrBut to mice. The ability of compositions of the disclosure to exit the gut lumen and epithelium and enter the systemic circulation is key in providing butyrate to the depths of the gut wall and the gut-draining lymph nodes to prevent and treat IBD.

### C. Multiple Sclerosis (MS) and Other Neuroinflammatory Conditions

MS is an autoimmune disease that T cells are reactive to myelin autoantigens, resulting in a chronic demyelinating inflammation of the central nervous system (CNS). Studies have revealed that both genetic and environmental factors are involved in the development of this disease. In the inventors' biodistribution study, they showed that SerBut has significantly increased butyrate level in both brain and spinal cord, compared to SPF mice treated with PBS, and sodium butyrate, suggesting that SerBut may have potential in treating MS. Other neuroinflammatory conditions are also relevant for treatment, such as diabetic peripheral neuropathy, a condition that is a frequent complication of diabetes. Neuroinflammation is also involved in traumatic brain injury, and access of SerBut to the brain is of value there. The ability of compositions of the disclosure to exit the gut and enter the systemic circulation is key in providing butyrate to the nervous tissue throughout the body, both in the peripheral nerves as in peripheral neuropathy and in the CNS as in neuro-autoimmune disorders including MS, neuromyelitis optica, and others.

### D. Rheumatoid Arthritis (RA)

RA is a chronic systemic autoimmune disease affecting about 1 percent of the population worldwide. It can be caused by both genetic and environmental factors, including the effect from gut microbiota. Intestinal dysbiosis can increase sensitivity to arthritis through activation of autoreactive T cells in the gut. With the higher accumulation of SerBut and ThrBut into the mesenteric lymph nodes and spleen upon oral gavage, the immunomodulation effects from butyrate may be enhanced, thus the compounds and compositions of the disclosure may be used as a therapeutic for treating RA. The ability of compositions of the disclosure to exit the gut and enter the systemic circulation is key in providing butyrate to the both the joints and the joint-draining lymph nodes.

### E. Asthma

Asthma affects 8.0% of adults and 7.0% of children in the United State, according to CDC. Allergic asthma is characterized by airway hyperreactivity (AHR) mainly driven by aberrant Th2 responses. Type 2 innate lymphoid cells (ILC2s) are a critical source of Th2 cytokines including IL-5 and IL-13. Thio et al have shown that butyrate inhibited IL-5 and IL-13 production by murine ILC2s through HDAC inhibition, and systemic and local administration of butyrate ameliorated ILC2-driven AHR and airway inflammation. The inventors hypothesize that, by increasing the systemic butyrate level through delivery of the compositions and compounds of the disclosure, the butyrate would also have higher accumulation in the lung, thus show enhanced bioactivity or the immune modulation through those mechanism as described above. The ability of compositions of the disclosure to exit the gut and enter the systemic circulation is key in providing butyrate to the lung and the lung-draining lymph nodes for prevention and treatment of asthma, including allergic asthma.

### F. Celiac Disease (CeD)

Celiac disease is a common autoimmune disease characterized by small intestinal inflammation, destruction of intestinal villi, elongated crypts and disturbed epithelial barrier function from gluten ingestion. It is a major global public health problem, with the prevalence of 1.4% based on serological test or 0.7% based on biopsy confirmation worldwide. The susceptibility of the disease can be influenced by both genetic and environmental factors. It is hypothesized that oral treatment with compositions of the disclosure may prove useful in prevention and treatment of CeD. The ability of compositions of the disclosure to exit the gut and gut epithelium and enter the systemic circulation is key in providing butyrate to the depths of the gut wall and the gut-draining lymph nodes to prevent and treat CeD.

### G. Non-Alcoholic Steatohepatitis (NASH)

NASH is one of the most common liver diseases worldwide, and is commonly associated with obesity, insulin resistance, diabetes, and the metabolic syndrome. The cause of NASH can be a combination of genetic and environmental factors, but the exact underlying mechanism is still not well understood. Currently there is no FDA-approved treatment for NASH. Recent animal studies have suggested that butyrate may provide protective effects on liver damage of various etiologies, including NASH. Glucagon-like peptide-1 (GLP-1) agonists have shown therapeutic effects on non-alcoholic fatty liver disease (NAFLD) and NASH in animal models, but are lack of consistency in clinical studies in some NAFLD patients. Our PK study revealed that compounds and compositions of the disclosure had significantly higher accumulation to the liver even at 6 hr after oral administration, compared to sodium butyrate, suggesting that these amino acids conjugated to butyrate might have better potential efficacy on suppressing liver inflammation in the application of treating NASH. The ability of compositions of the disclosure to exit the gut and enter the systemic circulation and portal circulation is key in providing butyrate to the liver and liver-draining lymph nodes to prevent and treat liver inflammation, including NASH and NAFLD.

### H. Diabetes

Diabetes is an increasing cause of mortality and disease burden in the world. Type-1 diabetes (T1D) is a chronic autoimmune disease associated with islet-reactive T cell-mediated destruction of pancreatic beta cells. It results in insulin deficiency and hyperglycemia, and occurs mainly in children and young adults. While insulin administration is currently the best available treatment for T1D, it has several limitations including severe side effects and a risk of life-threatening hypoglycemia. Many studies suggest that a healthy gut microbiome-mediated immune modulation during is critical to prevent T1D development. Conversely, alterations in a microbiome composition can lead to a poorly educated and deregulated immunity, eventually causing the insulitis and the onset of T1D. The ability of compositions of the disclosure to exit the gut and enter the systemic circulation is key in providing butyrate to the pancreas and pancreas-draining lymph nodes to prevent development of diabetes including Type 1 diabetes.

### I. Alzheimer's Disease (AD)

Alzheimer's disease (AD) is estimated to affect 14 million adults in the United States by 2050. AD leads to progressive loss of memory, executive function, and eventual death. It has historically been characterized by the buildup of amyloid aggregates composed of amyloid beta plaques, neurofibrillary tangles composed of hyperphosphorylated Tau protein, and brain atrophy. Although there have been many strategies to clear amyloid and Tau, none have been approved for clinical use. Thus, there is a dire need for paradigm-altering therapeutics. It is apparent that the microbiota plays a role in Alzheimer's disease progression, and patients with a lower level of circulating butyrate have an increased buildup of amyloid pathology in the cortex. In addition to amyloid and Tau pathology, neuroinflammation has come into play as potential cause of pathology and neuronal cell death in AD. At the center of neuroinflammation is microglia, the resident macrophages of the brain. It has been demonstrated in primary rat microglia that butyrate treatment prevents secretion of inflammatory molecules, such as IL-6 and nitric oxide, by downregulating NF-κB. Thus, by acting to reduce plaques, enhance neurogenesis, and reverse neuroinflammation, butyrate holds promise in treating AD. The ability of compositions of the disclosure to exit the gut, enter the blood, and transit the BBB is key to prevent and treat AD.

### J. Inflammaging

Inflammaging is a chronic and low-grade inflammation that develops as one ages and contributes to the pathogenesis of age-related diseases. Various data suggest that besides persistent infections, cell debris, toxins and altered and misplaced self-molecules are major contributors to inflammaging. The gut microbiota, which undergoes profound remodeling with age, has an important influence on inflammation, as it is at the boundary between diet, metabolism and immune system. In fact, a growing amount of evidence suggests that microbiota of elderly people is enriched in pro-inflammatory commensals at the expense of beneficial, butyrate-producing microbes. The aging process is associated with a decline in autophagic capacity which impairs cellular housekeeping, leading to protein aggregation and accumulation of dysfunctional mitochondria which provoke reactive oxygen species (ROS) production and oxidative stress. Reversing or slowing inflammaging is a promising strategy to improve the healthy lifespan. HDAC inhibitors are thought, aside from lowering inflammation, to increase genomic stability, reduce protein aggregates, improve mitochondrial function, improve stem cell maintenance. The ability of compositions of the disclosure to exit the gut and enter the systemic circulation, to affect immune cells throughout the body including the secondary lymphoid organs is key to preventing inflammaging.

### K. β-Hemoglobinopathies

Thalassemia and sickle cell disease are inherited blood disorders, called β-hemoglobinopathies, that affect millions of people around the world. Thalassemia is caused by genetic mutations resulting in insufficient production of alpha- or beta-chain of the hemoglobin, resulting in children's chronic anemia, a hypermetabolic state, endocrine abnormalities and poor nutrition. In sickle cell disease, a single amino acid mutation on hemoglobin results in hemoglobin's altered properties—while normally functional when oxygenated, it forms insoluble polymers upon deoxygenation. The process deforms the red blood cells leading to their damage and short life time, leading to severe anemia. One of the strategies to maintain these blood diseases is to increase the production of fetal hemoglobin, which can decrease the degree of alpha-chain to beta-chain imbalance and protect from many of the complication of sickle cell disease and beta-thalassemia. The induction of fetal hemoglobin synthesis involved the epigenetic regulation of gene expression, which is mediated by HDAC inhibition. The ability of orally administered compositions of the disclosure to exit the gut to enter the blood and bone marrow, the site of hematopoiesis, is key to treating thalassemias and sickle cell disease.

### L. Atherosclerosis and Other Vascular Inflammatory Conditions

Cardiovascular disease associated with obesity and autoimmunity is the leading cause of death in the US. It is most commonly caused by atherosclerosis—a condition in which a chronic inflammation results in endothelial dysfunction. The accumulation of inflammatory cells, smooth muscle cells, lipids and connective tissue within the intima of the artery leads to a formation of fibrous plagues—a thickening deposits of fat that can block the circulating blood flow. Some of the plagues develop into forms that are vulnerable to rupture, causing thrombosis and stenosis. Immune response plays essential role in atherosclerosis development because oxidized cholesterol (oxLDL) activates endothelial cells and monocytes which triggers the inflammatory process. The most common treatment for atherosclerosis is surgery in which the plagues are removed and a proper blood flow is restored. Vascular surgery, however, sometimes leads to a serious complication—a development of neointimal hyperplasia, proliferation of smooth muscle cells and arterial restenosis (re-closing of blood vessels). The ability of the compositions of the disclosure to exit the gut to enter the blood circulation, to modulate vascular inflammation throughout the cardiovascular system, is key to prevent and treat atherosclerosis, stenosis, and restenosis.

### M. Atopic Dermatitis and Psoriasis

Atopic dermatitis and psoriasis are two prevalent inflammatory skin diseases. Atopic dermatitis, also called eczema, is accompanied with erythema, severe itching and hemorrhage and its causes include environmental changes, hyperimmune sensitivity, stress and genetic factors. It is mediated by mast cell degranulation and a type 2 helper T cells-mediated allergic disease. The pathogenesis of psoriasis is not as well understood, but it is thought to be influenced by genetic and immunological factors. Psoriasis is characterized by epidermal and dermal infiltration of activated T cells and epidermal keratinocytes hyperproliferation that presents itself as skin plaques and thickening of the skin. An imbalance of the gut microbiome may contribute to the development of psoriasis and eczema. The ability of compositions of the disclosure to exit the gut to enter the blood circulation to perfuse the microvasculature of the skin is key to prevent and treat atopic dermatitis and psoriasis.

### N. Pulmonary Fibrosis

Pulmonary fibrosis is a progressive and fatal lung disease which causes debilitating breathlessness and decline in lung function, ultimately learning to respiratory failure. Two main types of pulmonary fibrosis are idiopathic pulmonary fibrosis and cystic fibrosis. While the pathogenic mechanisms idiopathic pulmonary fibrosis are still under investigation, the current hypothesis is that repeated micro-injury of alveolar epithelial cells (by factors such as cigarette smoke, particulate dust, viral infections etc.) results in an exaggerated wound-healing response as well as infiltration with inflammatory cells and fibroblasts. The excessive deposition of extracellular matrix and formation of fibroblastic foci cause irreversible damage to the lung. In the case of cystic fibrosis, the disease is characterized by chronic inflammation resulting in recurrent infections of the respiratory system. It results in accumulation of mucus and breathing difficulty. Since dysregulation in the microbiome has been associated with the development of fibrosis, bacteria metabolite butyrate may be a promising therapeutic candidate. The ability of compositions of the disclosure to exit the gut to enter the blood circulation and thereby enter the lung though the lung's vasculature is key to prevent and treat pulmonary fibrosis.

### O. Systemic Sclerosis

Systemic sclerosis, also called scleroderma is a complex disease of unknown etiology that affects connective tissues in multiple organs. The disease manifests in abnormalities in immune system, both innate and adaptive, leading to production of autoantibodies and cell-mediated autoimmunity. Moreover, dysfunction of fibroblasts leads to excessive accumulation of collagen in the skin, blood vessels and internal organs. Some people suffering from scleroderma also exhibit microvascular endothelial cell/small vessel fibroproliferative vasculopathy. There is no cure for systemic sclerosis. Patients are provided with pain-relieving nonsteroidal anti-inflammatory drugs, a standard pharmacological treatment that does not modify the course of the disease. They are also prescribed anti-inflammatory diet, free of gluten, sugar and dairy and rich in fiber. Currently, there are several drugs in clinical trials aiming to reduce inflammation without disabling the immune system, including a small molecule Lenabasum, a selective agonist of the cannabinoid receptor type 2 (CB2)¹⁴⁷. Butyrate could potentially help mitigate the symptoms and progression of scleroderma, by acting directly on the immune system, lowering inflammation, improving Treg function and lowering antibody production. In addition, its demonstrated effects on fibroblasts differentiation, proliferation and collagen production could alleviate the fibrosis-related symptoms of scleroderma. The ability of the compositions of the disclosure to exit the gut to enter the systemic circulation to enter connective tissues throughout the body is key to prevent and treat systemic sclerosis.

### IV. Kits

Certain aspects of the disclosure also encompass kits for performing the methods of the disclosure, such as treatment of a disorder described herein. Aspects relate to kits comprising the therapeutic pharmaceutical compositions of the disclosure.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific therapeutic or non-therapeutic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

In some aspects, when the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

### V. Examples

The following examples are included to demonstrate preferred aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure. The Examples should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### Example 1: Oral Systemically-Bioavailable Butyrate Through Conjugation to Hydroxy-Amino Acids

### A. Synthesis and Characterization of Serine Butyrate (SerBut), Threonine Butyrate (ThrBut) and Tyrosine Butyrate (TyrBut)

### 1. Serine Butyrate (SerBut)

Alternative names: O-butyryl L-serine, L-serine butyrate

Formula: C₇H₁₃NO₄

Formula weight: 175.08 g/mol

Elem. Anal. (theor.): C, 47.99; H, 7.48; N, 8.00; O, 36.53;

¹H NMR (500 MHz, DMSO-d₆) [ppm]: 0.88 (3H, t), 1.55 (2H, m), 2.32 (2H, t), 4.30 (1H, t), 4.43 (2H, d), 8.66 (2H, s), 14.06 (1H, s)

### Structure of Serine Butyrate

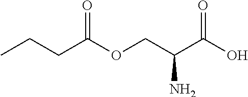

Synthesis method: L-Serine (20 g, 0.19 mol) was added to trifluoroacetic acid (200 ml) and the suspension was stirred for 30 min until everything dissolved. Butyryl chloride (25.7 ml, 0.23 mol) was then added to the solution and the mixture was stirred for 2 h at room temperature. The reaction was then transferred to an ice bath and diethyl ether (500 ml) was added which resulted in a precipitation of a white solid. The resultant fine white precipitate was collected by filtration, washed with cold diethyl ether, and dried under vacuum to afford 26.3 g of serine butyrate (0.15 mol, 79%).

### 2. Threonine Butyrate (ThrBut)

Alternative names: O-butyryl L-threonine, L-threonine butyrate

Formula: C₈H₁₅NO₄

Formula weight: 189.10 g/mol

Elem. Anal. (theor.): C, 50.78; H, 7.99; N, 7.40; O, 33.82;

¹H NMR (500 MHz, DMSO-d₆) [ppm]: 0.85 (3H, t), 1.30 (3H, d), 1.52 (2H, m), 2.26 (2H, t), 4.15 (1H, s), 5.25 (2H, m), 8.55 (2H, s), 14.02 (1H, s)

### Structure of Threonine Butyrate

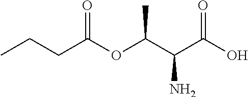

Synthesis method: L-Threonine (21.6 g, 0.19 mol) was added to trifluoroacetic acid (200 ml) and the suspension was stirred for 30 min until everything dissolved. Butyryl chloride (25.7 ml, 0.23 mol) was then added to the solution and the mixture was stirred for 2 h at room temperature. The reaction was then transferred to an ice bath and diethyl ether (500 ml) was added which resulted in a precipitation of a white solid. The resultant fine white precipitate was collected by filtration, washed with cold diethyl ether, and dried under vacuum to afford 34 g of threonine butyrate (0.18 mol, 95%).

### 3. Tyrosine Butyrate (TyrBut)

Alternative names: O-butyryl L-tyrosine, L-tyrosine butyrate

Formula: C₁₃H₁₇NO₄

Formula weight: 251.12 g/mol

Elem. Anal. (theor.): C, 62.14; H, 6.82; N, 5.57; O, 25.47;

¹H NMR (500 MHz, DMSO-d₆) [ppm]: 0.95 (3H, t), 1.64 (2H, m), 2.53 (2H, t), 3.15 (2H, d), 4.13 (1H, s), 7.06 (2H, d), 7.31 (2H, d), 8.53 (2H, s), 13.80 (1H, s)

### Structure of Tyrosine Butyrate

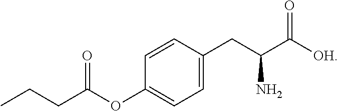 [SMILES: CCCC(=O)Oc1ccc(C[C@H](N)C(=O)O)cc1]

Synthesis method: L-Tyrosine (28.7 g, 0.19 mol) was added to trifluoroacetic acid (200 ml) and the suspension was stirred for 30 min until everything dissolved. Butyryl chloride (25.7 ml, 0.23 mol) was then added to the solution and the mixture was stirred for 2 h at room temperature. The reaction was then transferred to an ice bath and diethyl ether (500 ml) was added which resulted in a precipitation of a white solid. The resultant fine white precipitate was collected by filtration, washed with cold diethyl ether, and dried under vacuum to afford 40 g of tyrosine butyrate (0.16 mol, 84%).

### B. Biodistribution of SerBut and ThrBut

The small intestine is the primary site for the absorption of amino acids through amino acid transporters in intestinal epithelial cells (4). To evaluate whether the conjugation of amino acids to butyrate, i.e. SerBut and ThrBut in our case, enhances the absorption of butyrate through oral administration and increases the bioavailability, the inventors performed this biodistribution study and measured butyrate levels in plasma and several major organs after oral gavage of SerBut or ThrBut, and compared those with sodium butyrate (NaBut).

### 1. Methods

The specific-pathogen-free (SPF) female C57BL/6J mice were orally administered with 50.4 mg NaBut, 80 mg SerBut, 85.6 mg ThrBut (all containing equivalent 40 mg butyrate), or PBS. At 0.5 hr or 3 hr post administration, blood was collected by cheek bleeding. At 3 hr or 6 hr post administration, the mice were anesthetized under isoflurane and transcardially perfused with at least 30 ml phosphate-buffered saline (PBS) containing 1 mM EDTA. Organs including liver, mesenteric lymph nodes (mLNs), brain, spinal cord, and spleen were collected and frozen in dry ice immediately, and then transfer to −80° C. until further processing.

1:1 v/v acetonitrile (ACN) to water was used to extract butyrate from the plasma or organs. Plasma was mixed 1:1 with ACN/water solution, and then centrifuged to remove the denatured proteins. Organs were weighed and transferred to Lysing Matrix D tubes, and added with ACN/water 1:1 v/v solution followed by sample lysing using FastPrep-24 5G homogenizer (MP Biomedicals). The samples were then centrifuged, and the supernatant were collected for the measuring of butyrate.

Samples were prepared and derivatized as describe in the literature (5, 6). 3-nitrophenylhydrazine (NPH) stock solution was prepared at 0.02 M in water:acetonitrile 1:1 v/v. EDC stock solution (with 1% pyridine added) was prepared at 0.25 M in water:acetonitrile 1:1 v/v. 4-methylvaleric acid was added as internal standard. Samples were mixed with NPH stock and EDC stock at 1:1:1 ratio by volume. The mixture was heated by heating block at 60° C. for 30 min. Samples were then filtered through 0.22 m and transferred into HPLC vials and stored at 4° C. before analysis.

The Agilent 6460 Triple Quad MS-MS was used to detect the derivatized butyrate.

Both derivatized butyrate-NPH and 4-methylvaleric-NPH were detected in negative mode. Column: ThermoScientific C18 4.6×50 mm, 1.8 m particle size, at room temperature. Mobile phase A: water with 0.1% v/v formic acid. Mobile phase B: acetonitrile with 0.1% v/v formic acid. Injection volume: 5.0 μL. Flow rate: 0.5 mL/min. Gradient of solvent: 15% mobile phase B at 0.0 min; 100% mobile phase B at 3.5 min; 100% mobile phase B at 6.0 min; 15% mobile phase B at 6.5 min. The MS conditions were optimized on pure butyrate-NPH or 4-methylvaleric-NPH at 1 mM. The fragment voltage was 135 V and collision energy was set to 18 V. Multiple reaction monitoring (MRM) of 222→137 was assigned to butyrate, and MRM of 250→137 was assigned to 4-methylvaleric acid as internal standard. The ratio between MRM of butyrate and 4-methylvaleric acid was used to quantify the concentration of butyrate. The final butyrate content is each organ is normalized by the organ weight.

### 2. Results

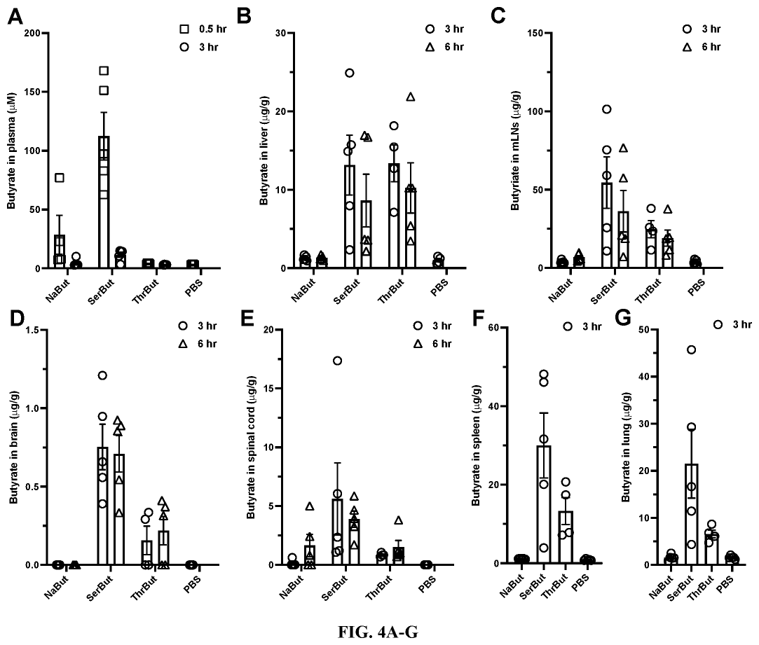

The inventors observed that the SerBut dramatically increased the butyrate level in the plasma at 0.5 hr after oral administration, although it dropped significantly after 3 hr (FIG. 4A). The NaBut or ThrBut did not show high level of butyrate in the plasma at those two time points. In the liver, where the orally taken drug would enter directly through the hepatic portal system, the inventors observed high level of butyrate from mice treated with both SerBut and ThrBut at both 3 hr and 6 hr post administration, but not from NaBut (FIG. 4B) SerBut and ThrBut also accumulates in the mesenteric LNs and high levels of butyrate were observed for both 3 hr and 6 hr time points (FIG. 4C), indicating the efficient lymphatic uptake of both SerBut and ThrBut in the gut. The higher level of butyrate in the spleen at 3 hr post-administration of SerBut as well as ThrBut also suggests that the circulating butyrate into into the secondary lymphoid organs ((FIG. 4F).

Some amino acids also across the blood-brain barrier (BBB), e.g. the L-serine across BBB via the sodium-dependent system A, and the sodium-independent alanine, serine, and cysteine system of transport (7,8). Montaser et al. demonstrated that utilizing L-type amino acid transporter 1 enhanced efficient brain delivery of small prodrugs across the blood-brain barrier. The inventors also investigated whether the amino acids would assist orally-administered butyrate to enter the central nervous system (CNS), including the brain and spinal cord. The inventors observed that the SerBut significantly enhanced the butyrate level both in brain and in spinal cord, compared to the mice treated with NaBut and ThrBut (FIG. 4D,E).

### Example 2: Administration of Serine Butyrate in a Multiple Sclerosis Mouse Model

### A. Method: Experimental Autoimmune Encephalomyelitis (EAE) Model

C57BL/6 female mice (8 weeks old) were purchased from Charles River Laboratories and housed in the animal facility at the University of Chicago for 2 weeks before immunization. All experiments were approved by the Institutional Animal Care and Use Committee of the University of Chicago

Female C57BL/6 mice at the age of week 10 were subcutaneously immunized at the dorsal flanks with an emulsion of MOG₃₅₋₅₅ in complete Freund's adjuvant (MOG₃₅₋₅₅/CFA Emulsion, Hooke Laboratories) on day 0, followed by i.p. administration of pertussis toxin in PBS on both day 0 and day 1. The mice were treated with PBS or serine butyrate (0.6 mg/g per dose) twice daily from day 2 to day 19. Another group of mice were treated with PBS once (in the morning) and serine butyrate (0.6 mg/g) once (in the afternoon) daily. The development of EAE was monitored and clinical scores were measured daily from day 7 to day 20. The clinical scores were determined based on the Hooke Laboratories scoring criteria (found online at hookelabs.com/services/cro/eae/MouseEAEscoring.html). In brief, EAE clinical scores ranged from a score of 0, corresponding to no obvious changes in motor function compared to non-immunized mice, to a score of 4, corresponding to limp tail and minimal or no movement of hind and/or fore limbs. On day 20, mice from the twice daily PBS or serine butyrate gavage groups were sacrificed. Their blood was collected through cardiac puncture, and spleen, spinal cord, mesenteric lymph nodes (LNs), central nervous system(CNS)-draining LNs (dLNs, including cervical LNs and iliac LNs) were harvested for the immunostaining followed by the flow cytometry analysis. The major cytokines from the plasma and spinal cord after homogenization were analyzed by the LegendPlex (BioLegend).

### B. Results

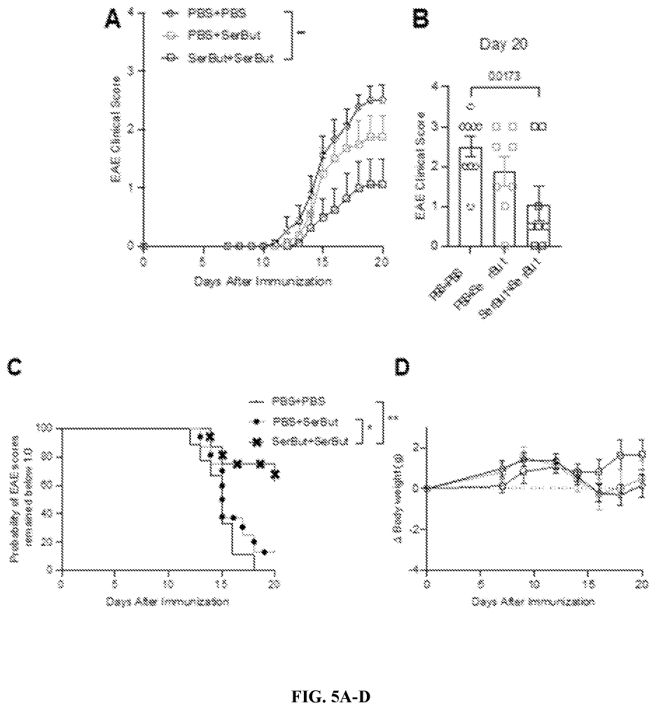

The inventors observed that twice daily gavages of serine butyrate significantly reduced the severity of EAE disease development, compared to the PBS treatment (FIG. 5). Our dose-dependent study also suggested that the 0.6 mg/g twice daily dose regimen of serine butyrate performed better than a single dose of serine butyrate. The majority of mice from the serine butyrate twice daily treated mice remained the clinical score below 1.0, suggesting an effective protection of disease progression from the treatment. The inventors also did not observe a significant body weight loss from the serine butyrate treated mice.

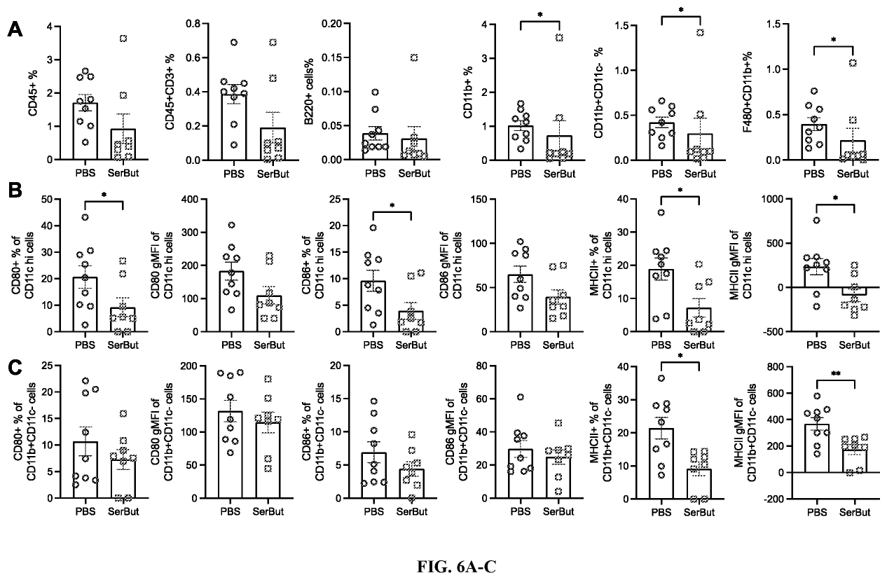

The inventors then analyzed immune cells in the spinal cord after treatment. The serine butyrate treatment significantly suppressed immune cell infiltration into the spinal cord (FIG. 6A), especially for the CD11b+, CD11b+CD11c−, and F4/80+CD11b+ cells. Those mice who were responsive to the treatment, as determined by their low EAE clinical scores, showed very few CD45+ immune cells, CD3+ T cells, B220+B cells, as well as those antigen presenting cells in the spinal cord. Furthermore, there was a significant reduction of CD80+% and CD86+% of CD11c^{hi} dendritic cells, as co-stimulatory markers, in the spinal cord of mice that treated with serine butyrate (FIG. 6B). The serine butyrate treatment was also associated with a similarly marked reduction of cell surface MHCII expression molecules in both CD11c^{hi} dendritic cells and CD11b+CD11c− monocytes (FIGS. 6B&C).

Since the serine butyrate was administered intragastrically and is expected to be absorbed in the intestine, the inventors also examined the effect of treatment on the myeloid cells in the mesenteric LNs. Similar to what the inventors observed in the spinal cord, there was a significant reduction of MHCII positive dendritic cells and macrophages from mice treated with serine butyrate. The MHCII expression as measured by the geometrical mean fluorescent intensity (gMFI) on those cells were also significantly lower from mice treated with serine butyrate, compared to PBS-treated mice.

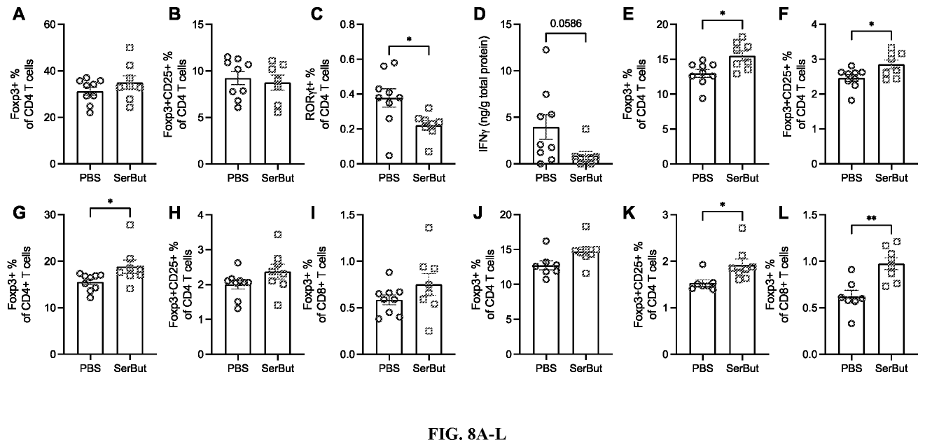

Next, the inventors examined T cells that were influenced by the serine butyrate treatment in this EAE model. The inventors observed less Th17 cells (RORγt+% of CD4+ T cells) presented in the spinal cord from serine butyrate-treated mice, compared to the PBS group (FIG. 8C). Those serine butyrate-treated mice also had minimal IFNγ in their spinal cord, while the PBS-treated mice had elevated levels of IFNγ (FIG. 8D). In the spleen, serine butyrate treatment significantly increased the regulatory T cells (Tregs), as indicated either by the percentage of Foxp3+ of CD4+ T cells, or the percentage of Foxp3+CD25+ of CD4+ T cells (FIGS. 8E&F). Similar induction of Tregs was observed in both CNS-draining LNs and mesenteric LNs (FIG. 8G-K). In addition, the inventors observed a significant induction of Foxp3+CD8+ Tregs in the mesenteric LNs (FIG. 8L).

In summary, the twice daily serine butyrate treatment (0.6 mg/g per dose) ameliorated EAE disease progression. This dose could be translated to a human dose of 0.05 g/kg considering the differences in body surface area. The serine butyrate decreased immune cell infiltration to the spinal cord, and suppressed the activation of antigen-presenting cells in both spinal cord and mesenteric LNs. In addition, the serine butyrate treatment reduced Th17 cells in the spinal cord, and induced Tregs in numerous lymphoid organs including spleen, CNS-draining and mesenteric LNs.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Hamer, H. M.; Jonkers, D.; Venema, K.; Vanhoutvin, S.; Troost, F. J.; Brummer, R. J., Review article: the role of butyrate on colonic function. Aliment Pharmacol Ther 2008, 27 (2), 104-19.
2. Boets, E.; Gomand, S. V.; Deroover, L.; Preston, T.; Vermeulen, K.; De Preter, V.; Hamer, H. M.; Van den Mooter, G.; De Vuyst, L.; Courtin, C. M.; Annaert, P.; Delcour, J. A.; Verbeke, K. A., Systemic availability and metabolism of colonic-derived short-chain fatty acids in healthy subjects: a stable isotope study. J Physiol 2017, 595 (2), 541-555.
3. Donohoe, D. R.; Garge, N.; Zhang, X.; Sun, W.; O'Connell, T. M.; Bunger, M. K.; Bultman, S. J., The microbiome and butyrate regulate energy metabolism and autophagy in the mammalian colon. Cell Metab 2011, 13 (5), 517-26.
4. Broer, S., Amino Acid Transport Across Mammalian Intestinal and Renal Epithelia. Physiological Reviews 2008, 88 (1), 249-286.
5. Torii, T.; Kanemitsu, K.; Wada, T.; Itoh, S.; Kinugawa, K.; Hagiwara, A., Measurement of short-chain fatty acids in human faeces using high-performance liquid chromatography: specimen stability. Ann Clin Biochem 2010, 47 (Pt 5), 447-52.
6. Tyagi, A.; Yu, M.; Darby, T.; Vaccaro, C.; Li, J.; Owens, J.; Hsu, E.; Adams, J.; Weitzmann, M.; Jones, R.; Pacifici, R., The Microbial Metabolite Butyrate Stimulates Bone Formation via T Regulatory Cell-Mediated Regulation of WNT10B Expression. Immunity 2018, 49 (6), 1116-+.
7. Hawkins, R. A.; O'Kane, R. L.; Simpson, I. A.; Vina, J. R., Structure of the Blood-Brain Barrier and Its Role in the Transport of Amino Acids. The Journal of Nutrition 2006, 136 (1), 218S-226S.
8. Tayarani, I.; Lefauconnier, J.-M.; Roux, F.; Bourre, J.-M., Evidence for an Alanine, Serine, and Cysteine System of Transport in Isolated Brain Capillaries. Journal of Cerebral Blood Flow & Metabolism 1987, 7 (5), 585-591.
9. Canani, R. B.; Costanzo, M. D.; Leone, L.; Bedogni, G.; Brambilla, P.; Cianfarani, S.; Nobili, V.; Pietrobelli, A.; Agostoni, C., Epigenetic mechanisms elicited by nutrition in early life. Nutr Res Rev 2011, 24 (2), 198-205.
10. Peng, L.; Li, Z. R.; Green, R. S.; Holzman, I. R.; Lin, J., Butyrate enhances the intestinal barrier by facilitating tight junction assembly via activation of AMP-activated protein kinase in Caco-2 cell monolayers. J Nutr 2009, 139 (9), 1619-25.
11. Peng, L.; He, Z.; Chen, W.; Holzman, I. R.; Lin, J., Effects of Butyrate on Intestinal Barrier Function in a Caco-2 Cell Monolayer Model of Intestinal Barrier. Pediatric Research 2007, 61 (1), 37-41.
12. Berni Canani, R.; Di Costanzo, M.; Leone, L., The epigenetic effects of butyrate: potential therapeutic implications for clinical practice. Clin Epigenetics 2012, 4 (1), 4-4.
13. McKay, J. A.; Mathers, J. C., Diet induced epigenetic changes and their implications for health. Acta Physiol (Oxf) 2011, 202 (2), 103-18.
14. Meijer, K.; de Vos, P.; Priebe, M. G., Butyrate and other short-chain fatty acids as modulators of immunity: what relevance for health? Curr Opin Clin Nutr Metab Care 2010, 13 (6), 715-21.
15. Kaisar, M. M. M.; Pelgrom, L. R.; van der Ham, A. J.; Yazdanbakhsh, M.; Everts, B., Butyrate Conditions Human Dendritic Cells to Prime Type 1 Regulatory T Cells via both Histone Deacetylase Inhibition and G Protein-Coupled Receptor 109A Signaling. Front Immunol 2017, 8, 1429.
16. Breuer, R. I.; Soergel, K. H.; Lashner, B. A.; Christ, M. L.; Hanauer, S. B.; Vanagunas, A.; Harig, J. M.; Keshavarzian, A.; Robinson, M.; Sellin, J. H.; Weinberg, D.; Vidican, D. E.; Flemal, K. L.; Rademaker, A. W., Short chain fatty acid rectal irrigation for left-sided ulcerative colitis: a randomised, placebo controlled trial. Gut 1997, 40 (4), 485-91.
17. Vernia, P.; Cittadini, M.; Caprilli, R.; Torsoli, A., Topical treatment of refractory distal ulcerative colitis with 5-ASA and sodium butyrate. Dig Dis Sci 1995, 40 (2), 305-7.
18. Breuer, R. I.; Buto, S. K.; Christ, M. L.; Bean, J.; Vernia, P.; Paoluzi, P.; Di Paolo, M. C.; Caprilli, R., Rectal irrigation with short-chain fatty acids for distal ulcerative colitis. Preliminary report. Dig Dis Sci 1991, 36 (2), 185-7.
19. Sher, G. D.; Ginder, G. D.; Little, J.; Yang, S.; Dover, G. J.; Olivieri, N. F., Extended therapy with intravenous arginine butyrate in patients with beta-hemoglobinopathies. N Engl J Med 1995, 332 (24), 1606-10.
20. Gupta, R. S.; Warren, C. M.; Smith, B. M.; Jiang, J.; Blumenstock, J. A.; Davis, M. M.; Schleimer, R. P.; Nadeau, K. C., Prevalence and Severity of Food Allergies Among US Adults. JAMA Netw Open 2019, 2 (1), e185630.
21. Mathewson, N. D.; Jenq, R.; Mathew, A. V.; Koenigsknecht, M.; Hanash, A.; Toubai, T.; Oravecz-Wilson, K.; Wu, S. R.; Sun, Y.; Rossi, C.; Fujiwara, H.; Byun, J.; Shono, Y.; Lindemans, C.; Calafiore, M.; Schmidt, T. M.; Honda, K.; Young, V. B.; Pennathur, S.; van den Brink, M.; Reddy, P., Gut microbiome-derived metabolites modulate intestinal epithelial cell damage and mitigate graft-versus-host disease. Nat Immunol 2016, 17 (5), 505-513.
22. Fachi, J. L.; Felipe, J. S.; Pral, L. P.; da Silva, B. K.; Corrêa, R. O.; de Andrade, M. C. P.; da Fonseca, D. M.; Basso, P. J.; Camara, N. O. S.; de Sales E Souza, É.; Dos Santos Martins, F.; Guima, S. E. S.; Thomas, A. M.; Setubal, J. C.; Magalhães, Y. T.; Forti, F. L.; Candreva, T.; Rodrigues, H. G.; de Jesus, M. B.; Consonni, S. R.; Farias, A. D. S.; Varga-Weisz, P.; Vinolo, M. A. R., Butyrate Protects Mice from Clostridium difficile-Induced Colitis through an HIF-1-Dependent Mechanism. Cell Rep 2019, 27 (3), 750-761.e7.
23. Furusawa, Y.; Obata, Y.; Fukuda, S.; Endo, T. A.; Nakato, G.; Takahashi, D.; Nakanishi, Y.; Uetake, C.; Kato, K.; Kato, T.; Takahashi, M.; Fukuda, N. N.; Murakami, S.; Miyauchi, E.; Hino, S.; Atarashi, K.; Onawa, S.; Fujimura, Y.; Lockett, T.; Clarke, J. M.; Topping, D. L.; Tomita, M.; Hori, S.; Ohara, O.; Morita, T.; Koseki, H.; Kikuchi, J.; Honda, K.; Hase, K.; Ohno, H., Commensal microbe-derived butyrate induces the differentiation of colonic regulatory T cells. Nature 2013, 504 (7480), 446-50.
24. Arpaia, N.; Campbell, C.; Fan, X.; Dikiy, S.; van der Veeken, J.; deRoos, P.; Liu, H.; Cross, J. R.; Pfeffer, K.; Coffer, P. J.; Rudensky, A. Y., Metabolites produced by commensal bacteria promote peripheral regulatory T-cell generation. Nature 2013, 504 (7480), 451-5.
25. Smith, P. M.; Howitt, M. R.; Panikov, N.; Michaud, M.; Gallini, C. A.; Bohlooly-Y, M.; Glickman, J. N.; Garrett, W. S., The microbial metabolites, short-chain fatty acids, regulate colonic Treg cell homeostasis. Science 2013, 341 (6145), 569-73.
26. Di Costanzo, M.; Aitoro, R.; Paparo, L.; Stefka, A. T.; Patton, T. J.; Nocerino, R.; Calignano, A.; Meli, R.; Mattace Raso, G.; Simeoli, R.; Guandalini, S.; Nagler, C.; Berni Canani, R., Oral administration of butyrate protects against sensitization to cow milk protein in a murine model of cow milk allergy. Digestive and Liver Disease 2014, 46, e89.
27. Vonk, M. M.; Blokhuis, B. R. J.; Diks, M. A. P.; Wagenaar, L.; Smit, J. J.; Pieters, R. H. H.; Garssen, J.; Knippels, L. M. J.; van Esch, B. C. A. M., Butyrate Enhances Desensitization Induced by Oral Immunotherapy in Cow's Milk Allergic Mice. Mediators Inflamm 2019, 2019, 9062537-9062537.
28. Tan, J.; McKenzie, C.; Vuillermin, Peter J.; Goverse, G.; Vinuesa, Carola G.; Mebius, Reina E.; Macia, L.; Mackay, Charles R., Dietary Fiber and Bacterial SCFA Enhance Oral Tolerance and Protect against Food Allergy through Diverse Cellular Pathways. Cell Reports 2016, 15 (12), 2809-2824.
29. Alatab, S.; Sepanlou, S. G.; Ikuta, K.; Vahedi, H.; Bisignano, C.; Safiri, S.; Sadeghi, A.; Nixon, M. R.; Abdoli, A.; Abolhassani, H.; Alipour, V.; Almadi, M. A. H.; Almasi-Hashiani, A.; Anushiravani, A.; Arabloo, J.; Atique, S.; Awasthi, A.; Badawi, A.; Baig, A. A. A.; Bhala, N.; Bijani, A.; Biondi, A.; Borzi, A. M.; Burke, K. E.; Carvalho, F.; Daryani, A.; Dubey, M.; Eftekhari, A.; Fernandes, E.; Fernandes, J. C.; Fischer, F.; Haj-Mirzaian, A.; Haj-Mirzaian, A.; Hasanzadeh, A.; Hashemian, M.; Hay, S. I.; Hoang, C. L.; Househ, M.; Ilesanmi, O. S.; Jafari Balalami, N.; James, S. L.; Kengne, A. P.; Malekzadeh, M. M.; Merat, S.; Meretoja, T. J.; Mestrovic, T.; Mirrakhimov, E. M.; Mirzaei, H.; Mohammad, K. A.; Mokdad, A. H.; Monasta, L.; Negoi, I.; Nguyen, T. H.; Nguyen, C. T.; Pourshams, A.; Poustchi, H.; Rabiee, M.; Rabiee, N.; Ramezanzadeh, K.; Rawaf, D. L.; Rawaf, S.; Rezaei, N.; Robinson, S. R.; Ronfani, L.; Saxena, S.; Sepehrimanesh, M.; Shaikh, M. A.; Sharafi, Z.; Sharif, M.; Siabani, S.; Sima, A. R.; Singh, J. A.; Soheili, A.; Sotoudehmanesh, R.; Suleria, H. A. R.; Tesfay, B. E.; Tran, B.; Tsoi, D.; Vacante, M.; Wondmieneh, A. B.; Zarghi, A.; Zhang, Z.-J.; Dirac, M.; Malekzadeh, R.; Naghavi, M., The global, regional, and national burden of inflammatory bowel disease in 195 countries and territories, 1990& #x2013; 2017: a systematic analysis for the Global Burden of Disease Study 2017. The Lancet Gastroenterology & Hepatology 2020, 5 (1), 17-30.
30. Thorburn, A. N.; Macia, L.; Mackay, C. R., Diet, metabolites, and “western-lifestyle” inflammatory diseases. Immunity 2014, 40 (6), 833-42.
31. Yu, L. C.-H., Microbiota dysbiosis and barrier dysfunction in inflammatory bowel disease and colorectal cancers: exploring a common ground hypothesis. Journal of Biomedical Science 2018, 25 (1), 79.
32. Chen, G.; Ran, X.; Li, B.; Li, Y.; He, D.; Huang, B.; Fu, S.; Liu, J.; Wang, W., Sodium Butyrate Inhibits Inflammation and Maintains Epithelium Barrier Integrity in a TNBS-induced Inflammatory Bowel Disease Mice Model. EBioMedicine 2018, 30, 317-325.
33. Chen, J.; Zhao, K.-N.; Vitetta, L., Effects of Intestinal Microbial-Elaborated Butyrate on Oncogenic Signaling Pathways. Nutrients 2019, 11 (5), 1026.
34. Handel, A. E.; Giovannoni, G.; Ebers, G. C.; Ramagopalan, S. V., Environmental factors and their timing in adult-onset multiple sclerosis. Nature Reviews Neurology 2010, 6 (3), 156-166.
35. Jangi, S.; Gandhi, R.; Cox, L. M.; Li, N.; von Glehn, F.; Yan, R.; Patel, B.; Mazzola, M. A.; Liu, S.; Glanz, B. L.; Cook, S.; Tankou, S.; Stuart, F.; Melo, K.; Nejad, P.; Smith, K.; Topguolu, B. D.; Holden, J.; Kivisakk, P.; Chitnis, T.; De Jager, P. L.; Quintana, F. J.; Gerber, G. K.; Bry, L.; Weiner, H. L., Alterations of the human gut microbiome in multiple sclerosis. Nature Communications 2016, 7 (1), 12015.
36. Cosorich, I.; Dalla-Costa, G.; Sorini, C.; Ferrarese, R.; Messina, M. J.; Dolpady, J.; Radice, E.; Mariani, A.; Testoni, P. A.; Canducci, F.; Comi, G.; Martinelli, V.; Falcone, M., High frequency of intestinal T&lt;sub&gt;H&lt;/sub&gt;17 cells correlates with microbiota alterations and disease activity in multiple sclerosis. Science Advances 2017, 3 (7), e1700492.
37. Swidsinski, A.; Dorffel, Y.; Loening-Baucke, V.; Gille, C.; Goktas, O.; Reiβhauer, A.; Neuhaus, J.; Weylandt, K.-H.; Guschin, A.; Bock, M., Reduced Mass and Diversity of the Colonic Microbiome in Patients with Multiple Sclerosis and Their Improvement with Ketogenic Diet. Frontiers in Microbiology 2017, 8 (1141).
38. Cekanaviciute, E.; Yoo, B. B.; Runia, T. F.; Debelius, J. W.; Singh, S.; Nelson, C. A.; Kanner, R.; Bencosme, Y.; Lee, Y. K.; Hauser, S. L.; Crabtree-Hartman, E.; Sand, I. K.; Gacias, M.; Zhu, Y.; Casaccia, P.; Cree, B. A. C.; Knight, R.; Mazmanian, S. K.; Baranzini, S. E., Gut bacteria from multiple sclerosis patients modulate human T cells and exacerbate symptoms in mouse models. Proceedings of the National Academy of Sciences 2017, 114 (40), 10713-10718.
39. Berer, K.; Gerdes, L. A.; Cekanaviciute, E.; Jia, X.; Xiao, L.; Xia, Z.; Liu, C.; Klotz, L.; Stauffer, U.; Baranzini, S. E.; Kumpfel, T.; Hohlfeld, R.; Krishnamoorthy, G.; Wekerle, H., Gut microbiota from multiple sclerosis patients enables spontaneous autoimmune encephalomyelitis in mice. Proceedings of the National Academy of Sciences 2017, 114 (40), 10719-10724.
40. Cantarel, B. L.; Waubant, E.; Chehoud, C.; Kuczynski, J.; DeSantis, T. Z.; Warrington, J.; Venkatesan, A.; Fraser, C. M.; Mowry, E. M., Gut Microbiota in Multiple Sclerosis. Possible Influence of Immunomodulators 2015, 63 (5), 729-734.
41. Stanisavljevid, S.; Dinid, M.; Jevtid, B.; Dedovid, N.; Momcilovid, M.; Dokid, J.; Golić, N.; Mostarica Stojković, M.; Miljković, D., Gut Microbiota Confers Resistance of Albino Oxford Rats to the Induction of Experimental Autoimmune Encephalomyelitis. Front Immunol 2018, 9 (942).
42. Miyake, S.; Kim, S.; Suda, W.; Oshima, K.; Nakamura, M.; Matsuoka, T.; Chihara, N.; Tomita, A.; Sato, W.; Kim, S.-W.; Morita, H.; Hattori, M.; Yamamura, T., Dysbiosis in the Gut Microbiota of Patients with Multiple Sclerosis, with a Striking Depletion of Species Belonging to Clostridia XIVa and IV Clusters. PLOS ONE 2015, 10 (9), e0137429.
43. Mizuno, M.; Noto, D.; Kaga, N.; Chiba, A.; Miyake, S., The dual role of short fatty acid chains in the pathogenesis of autoimmune disease models. PLOS ONE 2017, 12 (2), e0173032.
44. Haghikia, A.; Jorg, S.; Duscha, A.; Berg, J.; Manzel, A.; Waschbisch, A.; Hammer, A.; Lee, D.-H.; May, C.; Wilck, N.; Balogh, A.; Ostermann, Annika I.; Schebb, N. H.; Akkad, Denis A.; Grohme, Diana A.; Kleinewietfeld, M.; Kempa, S.; Thone, J.; Demir, S.; Müller, Dominik N.; Gold, R.; Linker, Ralf A., Dietary Fatty Acids Directly Impact Central Nervous System Autoimmunity via the Small Intestine. Immunity 2015, 43 (4), 817-829.
45. Chen, T.; Noto, D.; Hoshino, Y.; Mizuno, M.; Miyake, S., Butyrate suppresses demyelination and enhances remyelination. Journal of Neuroinflammation 2019, 16 (1), 165.
46. Nisha, V.; Marilyn, E. M., Role of Monocarboxylate Transporters in Drug Delivery to the Brain. Current Pharmaceutical Design 2014, 20 (10), 1487-1498.
47. Kekuda, R.; Manoharan, P.; Baseler, W.; Sundaram, U., Monocarboxylate 4 Mediated Butyrate Transport in a Rat Intestinal Epithelial Cell Line. Digestive Diseases and Sciences 2013, 58 (3), 660-667.
48. Silva, Y. P.; Bernardi, A.; Frozza, R. L., The Role of Short-Chain Fatty Acids From Gut Microbiota in Gut-Brain Communication. Frontiers in Endocrinology 2020, 11 (25).
49. Bachmann, C.; Colombo, J.-P.; Beruter, J., Short chain fatty acids in plasma and brain: Quantitative determination by gas chromatography. Clinica Chimica Acta 1979, 92 (2), 153-159.
50. Harris, E. D., Rheumatoid Arthritis. New England Journal of Medicine 1990, 322 (18), 1277-1289.
51. Horta-Baas, G.; Romero-Figueroa, M. d. S.; Montiel-Jarquin, A. J.; Pizano-Zirate, M. L.; Garcia-Mena, J.; Ramirez-Durin, N., Intestinal Dysbiosis and Rheumatoid Arthritis: A Link between Gut Microbiota and the Pathogenesis of Rheumatoid Arthritis. Journal of Immunology Research 2017, 2017, 4835189.
52. Maeda, Y.; Takeda, K., Role of Gut Microbiota in Rheumatoid Arthritis. Journal of Clinical Medicine 2017, 6 (6), 60.
53. Maeda, Y.; Kurakawa, T.; Umemoto, E.; Motooka, D.; Ito, Y.; Gotoh, K.; Hirota, K.; Matsushita, M.; Furuta, Y.; Narazaki, M.; Sakaguchi, N.; Kayama, H.; Nakamura, S.; Iida, T.; Saeki, Y.; Kumanogoh, A.; Sakaguchi, S.; Takeda, K., Dysbiosis Contributes to Arthritis Development via Activation of Autoreactive T Cells in the Intestine. Arthritis & Rheumatology 2016, 68 (11), 2646-2661.
54. Stoll, M. L.; Kumar, R.; Morrow, C. D.; Lefkowitz, E. J.; Cui, X.; Genin, A.; Cron, R. Q.; Elson, C. O., Altered microbiota associated with abnormal humoral immune responses to commensal organisms in enthesitis-related arthritis. Arthritis Res Ther 2014, 16 (6), 486-486.
55. Kim, D. S.; Kwon, J.-E.; Lee, S. H.; Kim, E. K.; Ryu, J.-G.; Jung, K.-A.; Choi, J.-W.; Park, M.-J.; Moon, Y.-M.; Park, S.-H.; Cho, M.-L.; Kwok, S.-K., Attenuation of Rheumatoid Inflammation by Sodium Butyrate Through Reciprocal Targeting of HDAC2 in Osteoclasts and HDAC8 in T Cells. Front Immunol 2018, 9, 1525-1525.
56. Rosser, E. C.; Piper, C. J. M.; Matei, D. E.; Blair, P. A.; Rendeiro, A. F.; Orford, M.; Alber, D. G.; Krausgruber, T.; Catalan, D.; Klein, N.; Manson, J. J.; Drozdov, I.; Bock, C.; Wedderburn, L. R.; Eaton, S.; Mauri, C., Microbiota-Derived Metabolites Suppress Arthritis by Amplifying Aryl-Hydrocarbon Receptor Activation in Regulatory B Cells. Cell Metabolism 2020, 31 (4), 837-851.e10.
57. Thio, C. L.-P.; Chi, P.-Y.; Lai, A. C.-Y.; Chang, Y.-J., Regulation of type 2 innate lymphoid cell-dependent airway hyperreactivity by butyrate. Journal of Allergy and Clinical Immunology 2018, 142 (6), 1867-1883.e12.
58. Cait, A.; Hughes, M. R.; Antignano, F.; Cait, J.; Dimitriu, P. A.; Maas, K. R.; Reynolds, L. A.; Hacker, L.; Mohr, J.; Finlay, B. B.; Zaph, C.; McNagny, K. M.; Mohn, W. W., Microbiome-driven allergic lung inflammation is ameliorated by short-chain fatty acids. Mucosal Immunology 2018, 11 (3), 785-795.
59. Lloyd, C. M.; Hawrylowicz, C. M., Regulatory T Cells in Asthma. Immunity 2009, 31 (3), 438-449.
60. Thorburn, A. N.; McKenzie, C. I.; Shen, S.; Stanley, D.; Macia, L.; Mason, L. J.; Roberts, L. K.; Wong, C. H. Y.; Shim, R.; Robert, R.; Chevalier, N.; Tan, J. K.; Marino, E.; Moore, R. J.; Wong, L.; McConville, M. J.; Tull, D. L.; Wood, L. G.; Murphy, V. E.; Mattes, J.; Gibson, P. G.; Mackay, C. R., Evidence that asthma is a developmental origin disease influenced by maternal diet and bacterial metabolites. Nature Communications 2015, 6 (1), 7320.
61. Theiler, A.; Barnthaler, T.; Platzer, W.; Richtig, G.; Peinhaupt, M.; Rittchen, S.; Kargl, J.; Ulven, T.; Marsh, L. M.; Marsche, G.; Schuligoi, R.; Sturm, E. M.; Heinemann, A., Butyrate ameliorates allergic airway inflammation by limiting eosinophil trafficking and survival. Journal of Allergy and Clinical Immunology 2019, 144 (3), 764-776.
62. Schulzke, J. D.; Bentzel, C. J.; Schulzke, I.; Riecken, E. O.; Fromm, M., Epithelial tight junction structure in the jejunum of children with acute and treated celiac sprue. Pediatr Res 1998, 43 (4 Pt 1), 435-41.
63. Schuppan, D.; Junker, Y.; Barisani, D., Celiac disease: from pathogenesis to novel therapies. Gastroenterology 2009, 137 (6), 1912-33.
64. Singh, P.; Arora, A.; Strand, T. A.; Leffler, D. A.; Catassi, C.; Green, P. H.; Kelly, C. P.; Ahuja, V.; Makharia, G. K., Global Prevalence of Celiac Disease: Systematic Review and Meta-analysis. Clin Gastroenterol Hepatol 2018, 16 (6), 823-836.e2.
65. Sellitto, M.; Bai, G.; Serena, G.; Fricke, W. F.; Sturgeon, C.; Gajer, P.; White, J. R.; Koenig, S. S.; Sakamoto, J.; Boothe, D.; Gicquelais, R.; Kryszak, D.; Puppa, E.; Catassi, C.; Ravel, J.; Fasano, A., Proof of concept of microbiome-metabolome analysis and delayed gluten exposure on celiac disease autoimmunity in genetically at-risk infants. PLoS One 2012, 7 (3), e33387.
66. Chander, A. M.; Yadav, H.; Jain, S.; Bhadada, S. K.; Dhawan, D. K., Cross-Talk Between Gluten, Intestinal Microbiota and Intestinal Mucosa in Celiac Disease: Recent Advances and Basis of Autoimmunity. Frontiers in microbiology 2018, 9, 2597-2597.
67. Olivares, M.; Neef, A.; Castillejo, G.; Palma, G. D.; Varea, V.; Capilla, A.; Palau, F.; Nova, E.; Marcos, A.; Polanco, I.; Ribes-Koninckx, C.; Ortigosa, L.; Izquierdo, L.; Sanz, Y., The HLA-DQ2 genotype selects for early intestinal microbiota composition in infants at high risk of developing coeliac disease. Gut 2015, 64 (3), 406-17.
68. Pozo-Rubio, T.; de Palma, G.; Mujico, J. R.; Olivares, M.; Marcos, A.; Acuna, M. D.; Polanco, I.; Sanz, Y.; Nova, E., Influence of early environmental factors on lymphocyte subsets and gut microbiota in infants at risk of celiac disease; the PROFICEL study. Nutr Hosp 2013, 28 (2), 464-73.
69. Shmidt, E.; Smyrk, T. C.; Boswell, C. L.; Enders, F. T.; Oxentenko, A. S., Increasing duodenal intraepithelial lymphocytosis found at upper endoscopy: time trends and associations. Gastrointest Endosc 2014, 80 (1), 105-11.
70. Round, J. L.; Mazmanian, S. K., The gut microbiota shapes intestinal immune responses during health and disease. Nat Rev Immunol 2009, 9 (5), 313-23.
71. Cook, L.; Munier, C. M. L.; Seddiki, N.; van Bockel, D.; Ontiveros, N.; Hardy, M. Y.; Gillies, J. K.; Levings, M. K.; Reid, H. H.; Petersen, J.; Rossjohn, J.; Anderson, R. P.; Zaunders, J. J.; Tye-Din, J. A.; Kelleher, A. D., Circulating gluten-specific FOXP3(+)CD39(+) regulatory T cells have impaired suppressive function in patients with celiac disease. J Allergy Clin Immunol 2017, 140 (6), 1592-1603.e8.
72. Rosso, C.; Mezzabotta, L.; Gaggini, M.; Salomone, F.; Gambino, R.; Marengo, A.; Saba, F.; Vanni, E.; Younes, R.; Saponaro, C.; Buzzigoli, E.; Caviglia, G. P.; Abate, M. L.; Smedile, A.; Rizzetto, M.; Cassader, M.; Gastaldelli, A.; Bugianesi, E., Peripheral insulin resistance predicts liver damage in nondiabetic subjects with nonalcoholic fatty liver disease. Hepatology 2016, 63 (1), 107-116.
73. Sun, J.; Wu, Q.; Sun, H.; Qiao, Y., Inhibition of histone deacetylase by butyrate protects rat liver from ischemic reperfusion injury. Int J Mol Sci 2014, 15 (11), 21069-79.
74. Qiao, Y. L.; Qian, J. M.; Wang, F. R.; Ma, Z. Y.; Wang, Q. W., Butyrate protects liver against ischemia reperfusion injury by inhibiting nuclear factor kappa B activation in Kupffer cells. J Surg Res 2014, 187 (2), 653-9.
75. Mattace Raso, G.; Simeoli, R.; Russo, R.; Iacono, A.; Santoro, A.; Paciello, O.; Ferrante, M. C.; Canani, R. B.; Calignano, A.; Meli, R., Effects of Sodium Butyrate and Its Synthetic Amide Derivative on Liver Inflammation and Glucose Tolerance in an Animal Model of Steatosis Induced by High Fat Diet. PLOS ONE 2013, 8 (7), e68626.
76. Jin, C. J.; Sellmann, C.; Engstler, A. J.; Ziegenhardt, D.; Bergheim, I., Supplementation of sodium butyrate protects mice from the development of non-alcoholic steatohepatitis (NASH). British Journal of Nutrition 2015, 114 (11), 1745-1755.
77. Ye, J.; Lv, L.; Wu, W.; Li, Y.; Shi, D.; Fang, D.; Guo, F.; Jiang, H.; Yan, R.; Ye, W.; Li, L., Butyrate Protects Mice Against Methionine-Choline-Deficient Diet-Induced Non-alcoholic Steatohepatitis by Improving Gut Barrier Function, Attenuating Inflammation and Reducing Endotoxin Levels. Frontiers in Microbiology 2018, 9 (1967).
78. Zhou, D.; Chen, Y.-W.; Zhao, Z.-H.; Yang, R.-X.; Xin, F.-Z.; Liu, X.-L.; Pan, Q.; Zhou, H.; Fan, J.-G., Sodium butyrate reduces high-fat diet-induced non-alcoholic steatohepatitis through upregulation of hepatic GLP-1R expression. Experimental & Molecular Medicine 2018, 50 (12), 1-12.
79. Katsarou, A.; Gudbjornsdottir, S.; Rawshani, A.; Dabelea, D.; Bonifacio, E.; Anderson, B. J.; Jacobsen, L. M.; Schatz, D. A.; Lernmark, A. Type 1 Diabetes Mellitus. Nature Reviews Disease Primers 2017, 3 (1), 1-17.
80. Grunberger, G. The Need for Better Insulin Therapy. Diabetes, Obesity and Metabolism 2013, 15 (s1), 1-5.
81. Zheng, P.; Li, Z.; Zhou, Z. Gut Microbiome in Type 1 Diabetes: A Comprehensive Review. Diabetes Metab Res Rev 2018, 34 (7).
82. Zhou, H.; Sun, L.; Zhang, S.; Zhao, X.; Gang, X.; Wang, G. Evaluating the Causal Role of Gut Microbiota in Type 1 Diabetes and Its Possible Pathogenic Mechanisms. Front. Endocrinol. 2020, 11.
83. Vaarala, O. Gut Microbiota and Type 1 Diabetes. Rev Diabet Stud 2012, 9 (4), 251-259.
84. de Goffau, M. C.; Luopaj arvi, K.; Knip, M.; Ilonen, J.; Ruohtula, T.; Harkonen, T.; Orivuori, L.; Hakala, S.; Welling, G. W.; Harmsen, H. J.; Vaarala, O. Fecal Microbiota Composition Differs between Children with β-Cell Autoimmunity and Those Without. Diabetes 2013, 62 (4), 1238-1244.
85. Jacob, N.; Jaiswal, S.; Maheshwari, D.; Nallabelli, N.; Khatri, N.; Bhatia, A.; Bal, A.; Malik, V.; Verma, S.; Kumar, R.; Sachdeva, N. Butyrate Induced Tregs Are Capable of Migration from the GALT to the Pancreas to Restore Immunological Tolerance during Type-1 Diabetes. Sci Rep 2020, 10 (1), 19120.
86. Christensen, D. P.; Dahllof, M.; Lundh, M.; Rasmussen, D. N.; Nielsen, M. D.; Billestrup, N.; Grunnet, L. G.; Mandrup-Poulsen, T. Histone Deacetylase (HDAC) Inhibition as a Novel Treatment for Diabetes Mellitus. Mol Med 2011, 17 (5), 378-390.
87. Khan, S.; Jena, G. B. Protective Role of Sodium Butyrate, a HDAC Inhibitor on Beta-Cell Proliferation, Function and Glucose Homeostasis through Modulation of P38/ERK MAPK and Apoptotic Pathways: Study in Juvenile Diabetic Rat. Chemico-Biological Interactions 2014, 213, 1-12.
88. Hebert, L. E.; Weuve, J.; Scherr, P. A.; Evans, D. A. Alzheimer Disease in the United States (2010-2050) Estimated Using the 2010 Census. Neurology 2013, 80 (19), 1778-1783.
89. Marizzoni, M.; Cattaneo, A.; Mirabelli, P.; Festari, C.; Lopizzo, N.; Nicolosi, V.; Mombelli, E.; Mazzelli, M.; Luongo, D.; Naviglio, D.; Coppola, L.; Salvatore, M.; Frisoni, G. B. Short-Chain Fatty Acids and Lipopolysaccharide as Mediators Between Gut Dysbiosis and Amyloid Pathology in Alzheimer's Disease. J Alzheimers Dis 2020, 78 (2), 683-697.
90. Govindarajan, N.; Agis-Balboa, R. C.; Walter, J.; Sananbenesi, F.; Fischer, A. Sodium Butyrate Improves Memory Function in an Alzheimer's Disease Mouse Model When Administered at an Advanced Stage of Disease Progression. J Alzheimers Dis 2011, 26 (1), 187-197.
91. Fernando, W. M. A. D. B.; Martins, I. J.; Morici, M.; Bharadwaj, P.; Rainey-Smith, S. R.; Lim, W. L. F.; Martins, R. N. Sodium Butyrate Reduces Brain Amyloid-O Levels and Improves Cognitive Memory Performance in an Alzheimer's Disease Transgenic Mouse Model at an Early Disease Stage. J Alzheimers Dis 2020, 74 (1), 91-99.
92. Yoo, D. Y.; Kim, W.; Nam, S. M.; Kim, D. W.; Chung, J. Y.; Choi, S. Y.; Yoon, Y. S.; Won, M.-H.; Hwang, I K. Synergistic Effects of Sodium Butyrate, a Histone Deacetylase Inhibitor, on Increase of Neurogenesis Induced by Pyridoxine and Increase of Neural Proliferation in the Mouse Dentate Gyrus. Neurochem Res 2011, 36 (10), 1850-1857.
93. Huuskonen, J.; Suuronen, T.; Nuutinen, T.; Kyrylenko, S.; Salminen, A. Regulation of Microglial Inflammatory Response by Sodium Butyrate and Short-Chain Fatty Acids. Br J Pharmacol 2004, 141 (5), 874-880.
94. Franceschi, C.; Garagnani, P.; Parini, P.; Giuliani, C.; Santoro, A. Inflammaging: A New Immune-Metabolic Viewpoint for Age-Related Diseases. Nature Reviews Endocrinology 2018, 14 (10), 576-590.
95. Ragonnaud, E.; Biragyn, A. Gut Microbiota as the Key Controllers of “Healthy” Aging of Elderly People. Immunity & Ageing 2021, 18 (1), 2.
96. Biagi, E.; Nylund, L.; Candela, M.; Ostan, R.; Bucci, L.; Pini, E.; Nikkila, J.; Monti, D.; Satokari, R.; Franceschi, C.; Brigidi, P.; Vos, W. D. Through Ageing, and Beyond: Gut Microbiota and Inflammatory Status in Seniors and Centenarians. PLOS ONE 2010, 5 (5), e10667.
97. Salminen, A.; Kaarniranta, K.; Kauppinen, A. Inflammaging: Disturbed Interplay between Autophagy and Inflammasomes. Aging 2012, 4 (3), 166-175.
98. McIntyre, R. L.; Daniels, E. G.; Molenaars, M.; Houtkooper, R. H.; Janssens, G. E. From Molecular Promise to Preclinical Results: HDAC Inhibitors in the Race for Healthy Aging Drugs. EMBO Molecular Medicine 2019, 11 (9), e9854.
99. Lee, J. S.; Lee, G. M. Effect of Sodium Butyrate on Autophagy and Apoptosis in Chinese Hamster Ovary Cells. Biotechnol Prog 2012, 28 (2), 349-357.
100. El-Shorbagy, H. M. Potential Anti-Genotoxic Effect of Sodium Butyrate to Modulate Induction of DNA Damage by Tamoxifen Citrate in Rat Bone Marrow Cells. Cytotechnology 2017, 69 (1), 89-102.
101. Rose, S.; Bennuri, S. C.; Davis, J. E.; Wynne, R.; Slattery, J. C.; Tippett, M.; Delhey, L.; Melnyk, S.; Kahler, S. G.; MacFabe, D. F.; Frye, R. E. Butyrate Enhances Mitochondrial Function during Oxidative Stress in Cell Lines from Boys with Autism. Translational Psychiatry 2018, 8 (1), 1-17.
102. Walsh, M. E.; Bhattacharya, A.; Sataranatarajan, K.; Qaisar, R.; Sloane, L.; Rahman, M. M.; Kinter, M.; Van Remmen, H. The Histone Deacetylase Inhibitor Butyrate Improves Metabolism and Reduces Muscle Atrophy during Aging. Aging Cell 2015, 14 (6), 957-970.
103. Chonat, S.; Quinn, C. T. Current Standards of Care and Long Term Outcomes for Thalassemia and Sickle Cell Disease. Adv Exp Med Biol 2017, 1013, 59-87.
104. Bank, A. Understanding Globin Regulation in β-Thalassemia: It's as Simple as α, β, γ, δ. J Clin Invest 2005, 115 (6), 1470-1473.
105. Sundd, P.; Gladwin, M. T.; Novelli, E. M. Pathophysiology of Sickle Cell Disease. Annu Rev Pathol 2019, 14, 263-292.
106. Sher, G. D.; Ginder, G. D.; Little, J.; Yang, S.; Dover, G. J.; Olivieri, N. F. Extended Therapy with Intravenous Arginine Butyrate in Patients with β-Hemoglobinopathies. New England Journal of Medicine 1995, 332 (24), 1606-1610.
107. Burns, L. J.; Glauber, J. G.; Ginder, G. D. Butyrate Induces Selective Transcriptional Activation of a Hypomethylated Embryonic Globin Gene in Adult Erythroid Cells. Blood 1988, 72 (5), 1536-1542.
108. Patthamalai, P.; Fuchareon, S.; Chaneiam, N.; Ghalie, R. G.; Chui, D. H. K.; Boosalis, M. S.; Perrine, S. P. A Phase 2 Trial of HQK-1001 in HbE-β Thalassemia Demonstrates HbF Induction and Reduced Anemia. Blood 2014, 123 (12), 1956-1957.
109. Liakopoulou, E.; Blau, C. A.; Li, Q.; Josephson, B.; Wolf, J. A.; Fournarakis, B.; Raisys, V.; Dover, G.; Papayannopoulou, T.; Stamatoyannopoulos, G. Stimulation of Fetal Hemoglobin Production by Short Chain Fatty Acids. Blood 1995, 86 (8), 3227-3235.
110. Faller, D. V.; Perrine, S. P. Butyrate in the Treatment of Sickle Cell Disease and Beta-Thalassemia. Curr Opin Hematol 1995, 2 (2), 109-117.
111. Atweh, G. F.; Sutton, M.; Nassif, I.; Boosalis, V.; Dover, G. J.; Wallenstein, S.; Wright, E.; McMahon, L.; Stamatoyannopoulos, G.; Faller, D. V.; Perrine, S. P. Sustained Induction of Fetal Hemoglobin by Pulse Butyrate Therapy in Sickle Cell Disease. Blood 1999, 93 (6), 1790-1797.
112. Insull, W. The Pathology of Atherosclerosis: Plaque Development and Plaque Responses to Medical Treatment. The American Journal of Medicine 2009, 122 (1), S3-S14.
113. Oikonomou, E.; Leopoulou, M.; Theofilis, P.; Antonopoulos, A. S.; Siasos, G.; Latsios, G.; Mystakidi, V. C.; Antoniades, C.; Tousoulis, D. A Link between Inflammation and Thrombosis in Atherosclerotic Cardiovascular Diseases: Clinical and Therapeutic Implications. Atherosclerosis 2020, 309, 16-26.
114. Haudenschild, C. C. Morphology of Intimal Hyperplasia: Research Initiatives in Vascular Surgery *. Journal of Vascular Surgery 1989, 10 (5), 591-592.
115. Xiao, Y.; Guo, Z.; Li, Z.; Ling, H.; Song, C. Role and Mechanism of Action of Butyrate in Atherosclerotic Diseases: A Review. Journal of Applied Microbiology n/a (n/a).
116. Chen, W.; Zhang, S.; Wu, J.; Ye, T.; Wang, S.; Wang, P.; Xing, D. Butyrate-Producing Bacteria and the Gut-Heart Axis in Atherosclerosis. Clinica Chimica Acta 2020, 507, 236-241.
117. Troseid, M.; Andersen, G. O.; Broch, K.; Hov, J. R. The Gut Microbiome in Coronary Artery Disease and Heart Failure: Current Knowledge and Future Directions. EBioMedicine 2020, 52.
118. Kasahara, K.; Krautkramer, K. A.; Org, E.; Romano, K. A.; Kerby, R. L.; Vivas, E. I.; Mehrabian, M.; Denu, J. M.; Backhed, F.; Lusis, A. J.; Rey, F. E. Interactions between Roseburia Intestinalis and Diet Modulate Atherogenesis in a Murine Model. Nature Microbiology 2018, 3 (12), 1461-1471.
119. Aguilar, E. C.; Leonel, A. J.; Teixeira, L. G.; Silva, A. R.; Silva, J. F.; Pelaez, J. M. N.; Capettini, L. S. A.; Lemos, V. S.; Santos, R. a. S.; Alvarez-Leite, J. I. Butyrate Impairs Atherogenesis by Reducing Plaque Inflammation and Vulnerability and Decreasing NFκB Activation. Nutrition, Metabolism and Cardiovascular Diseases 2014, 24 (6), 606-613.
120. Aguilar, E. C.; Santos, L. C. dos; Leonel, A. J.; de Oliveira, J. S.; Santos, E. A.; Navia-Pelaez, J. M.; da Silva, J. F.; Mendes, B. P.; Capettini, L. S. A.; Teixeira, L. G.; Lemos, V. S.; Alvarez-Leite, J. I Oral Butyrate Reduces Oxidative Stress in Atherosclerotic Lesion Sites by a Mechanism Involving NADPH Oxidase Down-Regulation in Endothelial Cells. The Journal of Nutritional Biochemistry 2016, 34, 99-105.
121. Du, Y.; Li, X.; Su, C.; Xi, M.; Zhang, X.; Jiang, Z.; Wang, L.; Hong, B. Butyrate Protects against High-Fat Diet-Induced Atherosclerosis via up-Regulating ABCA1 Expression in Apolipoprotein E-Deficiency Mice. British Journal of Pharmacology 2020, 177 (8), 1754-1772.
122. Gao, F.; Lv, Y.-W.; Long, J.; Chen, J.-M.; He, J.; Ruan, X.-Z.; Zhu, H. Butyrate Improves the Metabolic Disorder and Gut Microbiome Dysbiosis in Mice Induced by a High-Fat Diet. Front. Pharmacol. 2019, 10.
123. Ho, K. J.; Xiong, L.; Hubert, N. J.; Nadimpalli, A.; Wun, K.; Chang, E. B.; Kibbe, M. R. Vancomycin Treatment and Butyrate Supplementation Modulate Gut Microbe Composition and Severity of Neointimal Hyperplasia after Arterial Injury. Physiol Rep 2015, 3 (12).
124. Nooromid Michael; Chen Edmund B.; Xiong Liqun; Shapiro Katherine; Jiang Qun; Demsas Falen; Eskandari Maeve; Priyadarshini Medha; Chang Eugene B.; Layden Brian T.; Ho Karen J. Microbe-Derived Butyrate and Its Receptor, Free Fatty Acid Receptor 3, But Not Free Fatty Acid Receptor 2, Mitigate Neointimal Hyperplasia Susceptibility After Arterial Injury. Journal of the American Heart Association 2020, 9 (13), e016235.
125. Zanvit, P.; Konkel, J. E.; Jiao, X.; Kasagi, S.; Zhang, D.; Wu, R.; Chia, C.; Ajami, N. J.; Smith, D. P.; Petrosino, J. F.; Abbatiello, B.; Nakatsukasa, H.; Chen, Q.; Belkaid, Y.; Chen, Z.-J.; Chen, W. Antibiotics in Neonatal Life Increase Murine Susceptibility to Experimental Psoriasis. Nat Commun 2015, 6, 8424.
126. Zikostelski, Z.; Milkovi, J.; Klimesovi, K.; Rossmann, P.; Hornovi, M.; Novosidova, I.; Stehlikovi, Z.; Kostovcik, M.; Hudcovic, T.; Stepinkovi, R.; Jnzlovi, K.; Hercogovi, J.; Tlaskalovi-Hogenovi, H.; Kverka, M. Intestinal Microbiota Promotes Psoriasis-Like Skin Inflammation by Enhancing Th17 Response. PLOS ONE 2016, 11 (7), e0159539.
127. Lee, S.-Y.; Lee, E.; Park, Y. M.; Hong, S.-J. Microbiome in the Gut-Skin Axis in Atopic Dermatitis. Allergy Asthma Immunol Res 2018, 10 (4), 354-362.
128. Hidalgo-Cantabrana, C.; Gómez, J.; Delgado, S.; Requena-López, S.; Queiro-Silva, R.; Margolles, A.; Coto, E.; Sanchez, B.; Coto-Segura, P. Gut Microbiota Dysbiosis in a Cohort of Patients with Psoriasis. Br J Dermatol 2019, 181 (6), 1287-1295.
129. Nylund, L.; Nermes, M.; Isolauri, E.; Salminen, S.; Vos, W. M. de; Satokari, R. Severity of Atopic Disease Inversely Correlates with Intestinal Microbiota Diversity and Butyrate-Producing Bacteria. Allergy 2015, 70 (2), 241-244.
130. Song, H.; Yoo, Y.; Hwang, J.; Na, Y.-C.; Kim, H. S. Faecalibacterium Prausnitzii Subspecies-Level Dysbiosis in the Human Gut Microbiome Underlying Atopic Dermatitis. J Allergy Clin Immunol 2016, 137 (3), 852-860.
131. Schwarz, A.; Philippsen, R.; Schwarz, T. Induction of Regulatory T Cells and Correction of Cytokine Disbalance by Short-Chain Fatty Acids: Implications for Psoriasis Therapy. Journal of Investigative Dermatology 2021, 141 (1), 95-104.e2.
132. Schwarz, A.; Bruhs, A.; Schwarz, T. The Short-Chain Fatty Acid Sodium Butyrate Functions as a Regulator of the Skin Immune System. Journal of Investigative Dermatology 2017, 137 (4), 855-864.
133. Krejner, A.; Bruhs, A.; Mrowietz, U.; Wehkamp, U.; Schwarz, T.; Schwarz, A. Decreased Expression of G-Protein-Coupled Receptors GPR43 and GPR109a in Psoriatic Skin Can Be Restored by Topical Application of Sodium Butyrate. Arch Dermatol Res 2018, 310 (9), 751-758.
134. Maher, T. M.; Strek, M. E. Antifibrotic Therapy for Idiopathic Pulmonary Fibrosis: Time to Treat. Respir Res 2019, 20 (1), 205.
135. Somogyi, V.; Chaudhuri, N.; Torrisi, S. E.; Kahn, N.; Müller, V.; Kreuter, M. The Therapy of Idiopathic Pulmonary Fibrosis: What Is Next? European Respiratory Review 2019, 28 (153).
136. Ho, K. J.; Varga, J. Early-Life Gut Dysbiosis: A Driver of Later-Life Fibrosis? Journal of Investigative Dermatology 2017, 137 (11), 2253-2255.
137. Frangoise, A.; Héry-Arnaud, G. The Microbiome in Cystic Fibrosis Pulmonary Disease. Genes (Basel) 2020, 11 (5).
138. Hewitt, R. J.; Molyneaux, P. L. The Respiratory Microbiome in Idiopathic Pulmonary Fibrosis. Ann Transl Med 2017, 5 (12).
139. Torii, K.; Maeshige, N.; Aoyama-Ishikawa, M.; Miyoshi, M.; Terashi, H.; Usami, M. Combination Therapy with Butyrate and Docosahexaenoic Acid for Keloid Fibrogenesis: An in Vitro Study. An Bras Dermatol 2017, 92 (2), 184-190.
140. Koga, Y.; Maeshige, N.; Tabuchi, H.; Uemura, M.; Aoyama-Ishikawa, M.; Miyoshi, M.; Katakami, C.; Usami, M. Suppression of Fibrosis in Human Pterygium Fibroblasts by Butyrate and Phenylbutyrate. Int J Ophthalmol 2017, 10 (9), 1337-1343.
141. Cantoni, S.; Galletti, M.; Zambelli, F.; Valente, S.; Ponti, F.; Tassinari, R.; Pasquinelli, G.; Galie, N.; Ventura, C. Sodium Butyrate Inhibits Platelet-Derived Growth Factor-Induced Proliferation and Migration in Pulmonary Artery Smooth Muscle Cells through Akt Inhibition. The FEBS Journal 2013, 280 (9), 2042-2055.
142. Kabel, A. M.; Omar, M. S.; Elmaaboud, M. A. A. Amelioration of Bleomycin-Induced Lung Fibrosis in Rats by Valproic Acid and Butyrate: Role of Nuclear Factor Kappa-B, Proinflammatory Cytokines and Oxidative Stress. International Immunopharmacology 2016, 39, 335-342.
143. Ghorbani, P.; Santhakumar, P.; Hu, Q.; Djiadeu, P.; Wolever, T. M. S.; Palaniyar, N.; Grasemann, H. Short-Chain Fatty Acids Affect Cystic Fibrosis Airway Inflammation and Bacterial Growth. European Respiratory Journal 2015, 46 (4), 1033-1045.
144. Vieira, R. de S.; Castoldi, A.; Basso, P. J.; Hiyane, M. I.; Camara, N. O. S.; Almeida, R. R. Butyrate Attenuates Lung Inflammation by Negatively Modulating Th9 Cells. Front. Immunol. 2019, 10.
145. Pattanaik, D.; Brown, M.; Postlethwaite, B. C.; Postlethwaite, A. E. Pathogenesis of Systemic Sclerosis. Front Immunol 2015, 6.
146. Scleroderma Treatment https://www.hopkinsmedicine.org/health/conditions-and-diseases/scleroderma/scleroderma-treatment (accessed Mar. 6, 2021).
147. Lenabasum https://www.corbuspharma.com/our-science/lenabasum (accessed Mar. 6, 2021).
148. Sanchez, H. N.; Moroney, J. B.; Gan, H.; Shen, T.; Im, J. L.; Li, T.; Taylor, J. R.; Zan, H.; Casali, P. B Cell-Intrinsic Epigenetic Modulation of Antibody Responses by Dietary Fiber-Derived Short-Chain Fatty Acids. Nature Communications 2020, 11 (1), 60.
149. Hussein, H.; Denanglaire, S.; Gool, F. V.; Azouz, A.; Ajouaou, Y.; El-Khatib, H.; Oldenhove, G.; Leo, O.; Andris, F. Multiple Environmental Signaling Pathways Control the Differentiation of RORγt-Expressing Regulatory T Cells. bioRxiv 2019, 791244.
150. Nastasi, C.; Fredholm, S.; Willerslev-Olsen, A.; Hansen, M.; Bonefeld, C. M.; Geisler, C.; Andersen, M. H.; Ødum, N.; Woetmann, A. Butyrate and Propionate Inhibit Antigen-Specific CD8+ T Cell Activation by Suppressing IL-12 Production by Antigen-Presenting Cells. Scientific Reports 2017, 7 (1)

1. A method for treating a disorder in a subject comprising administering an oral composition comprising a compound selected from 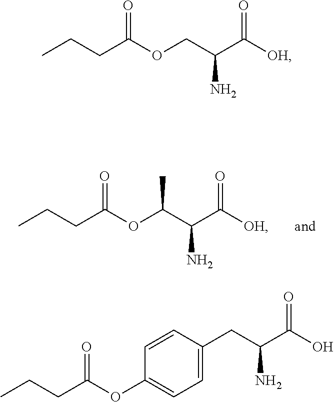 [SMILES: CCCC(=O)OC[C@H](N)C(=O)O ; CCCC(=O)O[C@@H](C)[C@H](N)C(=O)O ; "and" ; CCCC(=O)Oc1ccc(C[C@H](N)C(=O)O)cc1]
to the subject, wherein the disorder is selected from inflammatory bowel disease (IBD), multiple sclerosis (MS), a neuroinflammatory condition, food allergies, rheumatoid arthritis (RA), asthma, celiac disease, non-alcoholic steatohepatitis (NASH), diabetes, Alzheimer's disease (AD), inflammaging, beta-hemoglobinopathies, vascular inflammatory conditions, atopic dermatitis and psoriasis, pulmonary fibrosis, and systemic sclerosis.

2. The method of claim 1, wherein the compound comprises

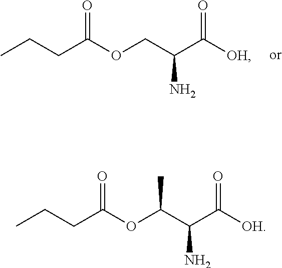

3. The method of claim 2, wherein the compound comprises

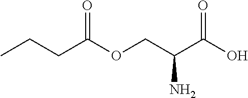

4. The method of any one of claims 1-3, wherein diabetes comprises Type I diabetes.

5. The method of claim 1-3, wherein the beta-hemoglobinopathies comprises thalassemia or sickle cell disease.

6. The method of claim 1-3, wherein the vascular inflammatory condition comprises atherosclerosis.

7. The method of claim 1-3, wherein the vascular inflammatory condition comprises restenosis

8. The method of claim 1-3, wherein the neuroinflammatory condition comprises diabetic peripheral neuropathy, neuroinflammation, neuroinflammation associated with traumatic brain injury, neuroinflammation associated with aging, neuroinflammation associated with spinal cord injury, neuroinflammation associated with infection, neuromyelitis optica, MS, Alzheimer's disease, or Parkinson's disease.

9. The method of any one of claims 1-8, wherein the compound is labile in vivo.

10. The method of any one of claims 1-9, wherein the method comprises administration of a systemically bioavailable composition.

11. The method of any one of claims 1-10, wherein the compound demonstrates significant biodistribution to the plasma, liver, mesenteric lymph nodes, brain, spinal cord, lung, and spleen over a control, wherein the control comprises sodium butyrate.

12. The method of any one of claims 1-11, wherein the method comprises administering a daily dose of the composition to the subject.

13. The method of claim 12, wherein the daily dose is administered for at least 30 days.

14. The method of any one of claims 1-13, wherein the subject comprises a human subject.

15. The method of claim 14, wherein the subject is 18 years old or more.

16. The method of any one of claims 12-15, wherein the composition comprises 2-8 grams of the compound in the daily dose.

17. The method of claim 16, wherein the composition comprises 3-6 grams of the compound in the daily dose.

18. The method of claim 17, wherein the composition comprises 6 grams of the compound in the daily dose.

19. The method of any one of claims 12-15, wherein the composition comprises 20-150 mg/kg of the compound in the daily dose.

20. The method of claim 19, wherein the composition comprises 50-100 mg/kg of the compound in the daily dose.

21. The method of claim 20, wherein the composition comprises 100 mg/kg of the compound in the daily dose.

22. The method of any one of claims 12-15, wherein the composition comprises less than 4 grams of the compound in the daily dose.

23. The method of any one of claims 1-22, wherein the oral composition comprises less than 4 grams of the compound.

24. The method of any one of claims 12-23, wherein the subject is administered the composition in at least two doses per day.

25. The method of claim 24, wherein the subject is administered the composition in at least two doses per day, wherein the total daily dose is 2-8 grams or 20-150 mg/kg of the compound.

26. The method of claim 25, wherein the subject is administered the composition in at least two doses per day, wherein the total daily dose is 3-6 grams or 50-100 mg/kg of the compound.

27. The method of claim 26, wherein the subject is administered the composition in at least two doses per day, wherein the total daily dose is 6 grams or 100 mg/kg of the compound.

28. The method of any one of claims 24-27, wherein the two doses are administered at a time period of at least 10 hours apart.

29. The method of any one of claims 1-28, wherein administration of the composition alleviates one or more symptoms of the disorder.

30. The method of any one of claims 1-29, wherein administration of the composition reduces inflammation in the subject.

31. The method of any one of claims 1-30, wherein the composition comprises a solution.

32. The method of any one of claims 1-30, wherein the composition comprises a tablet.

33. The method of claim 32, wherein the tablet comprises enteric coating and/or encapsulation of the compound.

34. The method of claim 32, wherein enteric coating and/or encapsulation of the compound is excluded.

35. The method of any one of claims 1-34, wherein the serum concentration of the compound 1-5 hours after administration is at least 50 nM.

36. The method of any one of claims 1-35, wherein the level of the compound in the liver, mesenteric lymph nodes, brain, spinal cord, lung, and/or spleen is estimated to be at least 2 μg/g.

37. The method of any one of claims 1-35, wherein the level of the compound in the liver, mesenteric lymph nodes, brain, spinal cord, lung, and/or spleen is estimated to be at least 10 μg/g.

38. A method for treating multiple sclerosis in a subject comprising administering an oral composition comprising 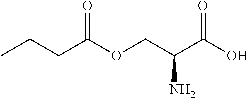
to the subject.

39. The method of claim 38, wherein the subject is a human subject.

40. The method of claim 39, wherein the subject is administered a total daily dose of 25 or 50 mg/kg.

41. The method of claim 40, wherein the subject is administered two doses per day, wherein the total daily dose is 25 or 50 mg/kg.

42. The method of claim 41, wherein the subject is administered two doses per day, wherein the total daily dose is 50 mg/kg.
